(19) United States
(12) Patent Application Publication
(10) Pub. No.: US 2025/0339362 A1
(43) Pub. Date: Nov. 6, 2025

(54) SKINCARE SUPPLEMENTS

(71) Applicant: Natals, Inc., Culver City, CA (US)

(72) Inventors: Nima ALAMDARI, Los Angeles, CA (US); Natasha CHAMBERLIN, Culver City, CA (US); Adeline M. GRIER-WELCH, Los Angeles, CA (US); Mastaneh SHARAFI, Redondo Beach, CA (US); Katerina SCHNEIDER, Los Angeles, CA (US); Arianne Y. VANCE, Santa Monica, CA (US)

(21) Appl. No.: 18/654,896

(22) Filed: May 3, 2024

(51) Int. Cl. A61K 8/73; A61K 8/11; A61K 8/42; A61K 8/60; A61K 8/67; A61K 8/9794; A61Q 19/00; A61Q 19/08

(52) U.S. Cl. CPC A61K 8/735; A61K 8/11; A61K 8/42; A61K 8/602; A61K 8/678; A61K 8/9794; A61Q 19/007; A61Q 19/08; A61K 2800/30; A61K 2800/87; A61K 2800/92

(57) ABSTRACT

The present disclosure provides compositions and dosage forms thereof, wherein the compositions comprise hyaluronic acid and a lipid extract comprising a ceramide and glycolipid. The present disclosure further provides methods for improving a skin property in a subject by administering the composition or dosage form to the subject.

### BACKGROUND

Skin aging occurs through slow degeneration of tissues as a result of intrinsic aging (e.g., effects of gravity, facial muscles acting on the skin, soft tissue loss, loss of tissue elasticity), as well as exposure to environmental factors during an individual's lifetime. The aging process is affected by such external and internal factors as ultraviolet exposure, nutrition, lifestyle, stress, and/or lack of sleep. The skin ages as the epidermis becomes thin, causing the junction with the dermis to flatten. Collagen present in the connective tissue of the dermis decreases, causing the skin to loosen and lose strength. As skin loses elasticity, it is less able to resist stretching. Coupled with gravity, muscle pull, and tissue changes, the skin begins to wrinkle. Water loss and breakdown of bonds between cells reduces the barrier function of the skin. The overall result of aging is progressive appearance of wrinkles, dermal atrophy, loss of hydration, loss of radiance, and degeneration of the elastic fiber network.

Skin is an important aspect of personal appearance, with many consumers having a desire to delay onset of dermatological signs of aging, or reduce the appearance of already existing dermatological signs of aging. Treatments ranging from topical creams to cosmetic surgery are available, but have downsides. For example, topical creams often have limited efficacy due to poor absorption and cosmetic surgeries are invasive and costly. Thus, there remains a need for skincare products that are economical and can be used without the negative effects of some currently known treatment modalities.

### SUMMARY

In some aspects, the disclosure provides a skincare supplement comprising an inner capsule and an outer capsule, wherein the inner capsule comprises a dose of hyaluronic acid (HA) having an average molecular weight of about 2 kDa to about 800 kDa, wherein the outer capsule comprises a dose of a lipid extract in an oil, wherein the lipid extract comprises a ceramide and a glycolipid, and wherein the inner capsule is contained in the outer capsule.

In some aspects, the disclosure provides a kit comprising a container comprising a skincare supplement described herein, and a package insert comprising instructions for administering the skincare supplement to a subject.

In some aspects, the disclosure provides a kit comprising a container comprising a skincare supplement described herein, and a package insert comprising instructions for administering the skincare supplement to a subject to improve a skin property. In some embodiments, the skin property is selected from (i) reduced fine lines and/or wrinkles; (ii) increased skin smoothness; (iii) increased skin hydration/moisturization; (iv) increased skin elasticity; (v) increased skin radiance; and (vi) a combination of (i)-(v).

In some embodiments of any of the foregoing or related aspects, the instructions further comprise administering to the subject a single skincare supplement once per day. In some embodiments, the instructions comprise administering the skincare supplement orally.

In some embodiments of any of the foregoing or related aspects, the skincare supplement is shelf-stable. In some embodiments, the kit comprises about 10 to about 100 skincare supplements. In some embodiments, the container comprises a desiccant. In some embodiments, the desiccant is contained in an insert. In some embodiments, the desiccant is contained in an inner wall of the container. In some embodiments, the desiccant comprises silica in an amount of about 3 grams to about 6 grams. In some embodiments, the desiccant comprises silica in an amount of about 5 grams to about 6 grams. In some embodiments, the container comprises a scented insert. In some embodiments, the scented insert comprises vanilla and a medium chain triglyceride.

In some aspects, the disclosure provides a method for improving a skin property in a human subject, comprising orally administering to the subject a skincare supplement in a dosing regimen of once per day for at least about 8 weeks, wherein the skincare supplement comprises a dose of HA and a dose of a lipid extract comprising a ceramide and a glycolipid. In some embodiments, the skin property is selected from (i) reduced fine lines and/or wrinkles; (ii) increased skin smoothness; (iii) increased skin hydration/moisturization; (iv) increased skin elasticity; (v) increased skin radiance; and (vi) a combination of (i)-(v). In some embodiments, the skin property is improved by about day 56, about day 63, about day 70, about day 77, or about day 84 of the dosing regimen as compared to a first day of the dosing regimen. In some embodiments, the improved skin property is maintained through a duration of the dosing regimen. In some embodiments, the skin property is improved at a site of a body of the subject, wherein the site is present in a facial region, a leg region, an arm region, a waist region, or a combination thereof.

In some aspects, the disclosure provides a method for delaying onset of dermatological sign of aging in a human subject, comprising orally administering to the subject a skincare supplement in a dosing regimen of once per day for at least about 8 weeks, wherein the skincare supplement comprises a dose of HA and a dose of a lipid extract comprising a ceramide and a glycolipid.

In some aspects, the disclosure provides a method of improving a dermatological sign of aging in a human subject, comprising orally administering to the subject a skincare supplement in a dosing regimen of once per day for at least about 8 weeks, wherein the skincare supplement comprises a dose of HA and a dose of a lipid extract comprising a ceramide and a glycolipid.

In some embodiments of any of the foregoing or related aspects, the dermatological sign of aging occurs at a site of a body of the subject, wherein the site is present in a facial region, a leg region, an arm region, a waist region, or a combination thereof. In some embodiments, the dermatological sign of aging is selected from wrinkles, fine lines, dryness, roughness, reduced elasticity, reduced radiance, and a combination thereof. In some embodiments, the dermatological sign of aging comprises wrinkles and/or fine lines. In some embodiments, the dermatological sign of aging comprises crow's feet wrinkles. In some embodiments, the dermatological sign of aging is improved or not worsened by at least about day 56, about day 63, about day 70, about day 77, or about day 84 of the dosing regimen as compared to a first day of the dosing regimen.

In some aspects, the disclosure provides a method for improving skin smoothness of a human subject, comprising orally administering to the subject a skincare supplement in a dosing regimen of once per day for at least about 8 weeks, wherein the skincare supplement comprises a dose of HA and a dose of a lipid extract comprising a ceramide and a glycolipid, and wherein skin smoothness is improved by at least about day 56 of the dosing regimen. In some embodiments, the skin smoothness is improved by at least about day 63, about day 70, about day 77, or about day 84 of the dosing regimen as compared to a first day of the dosing regimen. In some embodiments, an improvement to skin smoothness is maintained through at least about day 63, about day 70, about day 77, or about day 84 of the dosing regimen. In some embodiments, an improvement to skin smoothness is maintained through a duration of the dosing regimen. In some embodiments, the skin property is improved at a site of a body of the subject, wherein the site is present in a facial region, a leg region, an arm region, a waist region, or a combination thereof. In some embodiments, the site is present in a facial region. In some embodiments, the facial region comprises an eye, a forehead, a mouth, a nose, a cheek, an ear, or a combination thereof. In some embodiments, the site is present in a leg region. In some embodiments, the leg region comprises a thigh, a calf, a foot, or a combination thereof. In some embodiments, the site is present in an arm region. In some embodiments, the site is present in a waist region.

In some embodiments of any of the foregoing or related aspects, the skincare supplement comprises a capsule. In some embodiments, the capsule is an immediate release capsule. In some embodiments, the dose of HA and the dose of lipid extract are formulated together or separately. In some embodiments, the skincare supplement comprises an inner capsule contained in an outer capsule. In some embodiments, the inner capsule comprises the dose of HA and the outer capsule comprises the dose of lipid extract.

In some embodiments of any of the foregoing or related aspects, the dosing regimen is once per day for at least about 9 weeks, about 10 weeks, about 11 weeks, about 12 weeks, about 13 weeks, about 14 weeks, or about 15 weeks. In some embodiments, the dosing regimen is once per day for about 8 weeks to about 24 weeks. In some embodiments, the dosing regimen is once per day for about 8 weeks to about 12 weeks. In some embodiments, the human subject is an adult. In some embodiments, the human subject is about 18 years old to about 65 years old. In some embodiments, the human subject is male or female. In some embodiments, the human subject exhibits a dermatological sign of aging. In some embodiments, the supplement is essentially free of a component derived from an animal.

In some embodiments of any of the foregoing or related aspects, the HA has an average molecular weight effective for gastrointestinal permeation and/or absorption into skin after ingestion.

In some embodiments of any of the foregoing or related aspects, the HA comprises an average molecular weight of about 200 kDa to about 500 kDa. In some embodiments, the HA comprises an average molecular weight of about 300 kDa. In some embodiments, the dose of HA is about 25 mg to about 240 mg. In some embodiments, the dose of HA is about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, or about 240 mg. In some embodiments, the dose of HA is about 120 mg.

In some embodiments of any of the foregoing or related aspects, the dose of lipid extract is about 300 mg to about 400 mg. In some embodiments, the dose of lipid extract is about 300 mg to about 400 mg, or about 310 mg, about 320 mg, about 330 mg, about 340 mg, about 350 mg, about 360 mg, about 370 mg, about 380 mg, or about 390 mg. In some embodiments, the dose of lipid extract is about 350 mg. In some embodiments, the lipid extract comprises the ceramide and the glycolipid in a weight percent, wherein the weight percent of the ceramide is at least about 2%. In some embodiments, the weight percent of the ceramide is about 2%. In some embodiments, the weight percent of the glycolipid is at least about 15%. In some embodiments, the weight percent of the glycolipid is about 15%. In some embodiments, the glycolipid comprises digalactosyl diglyceride (DGDG). In some embodiments, the lipid extract is obtained from wheat. In some embodiments, the lipid extract is obtained from Triticum vulgare.

In some embodiments of any of the foregoing or related aspects, the inner capsule further comprises an excipient. In some embodiments, the excipient is selected from microcrystalline cellulose, ascorbyl palmitate, silicon dioxide, sunflower oil, and a combination thereof. In some embodiments, the oil comprises mixed tocopherols. In some embodiments, the inner capsule is a size 2 capsule. In some embodiments, the outer capsule is a size 00 capsule. In some embodiments, the inner capsule, the outer capsule, or both are an immediate release capsule. In some embodiments, the inner capsule and the outer capsule are an immediate release capsule. In some embodiments, the immediate release capsule comprises Hypromellose. In some embodiments, the inner capsule, the outer capsule, or both is translucent. In some embodiments, the inner capsule, the outer capsule, or both is semi-opaque or opaque.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1D are schematics showing the nested capsule design of an exemplary dietary supplement of the disclosure. The design includes an outer capsule and an inner capsule, the outer capsule containing a liquid wheat oil extract as a liquid and the inner capsule containing hyaluronic acid as a powdered solid. The exemplary dietary supplement is referred to as “HA/WEO supplement” herein.

FIGS. 2A-2B are graphs showing mean change in Crow's feet wrinkles at week 4, 8 and 12 (FIG. 2A) or at week 12 (FIG. 2B) as compared to week 0 in human subjects consuming the HA/WEO supplement once per day (n=31). The placebo group received a visually similar capsule (n=32).

FIGS. 3A-3D are images of the face of select subjects at week 0 (FIG. 3A or FIG. 3C) and week 12 (FIG. 3B or FIG. 3D) following daily consumption of the HA/WEO supplement. The inlays show a close-up image of the subject's crow's feet region.

FIGS. 4A-4B are graphs showing mean change in facial skin smoothness at week 4, 8 and 12 (FIG. 4B) or at week 12 (FIG. 4A) as compared to week 0 in human subjects consuming the HA/WEO supplement once per day (n=31). The placebo group received a visually similar capsule (n=32).

### DETAILED DESCRIPTION

The present disclosure relates, at least in part, to supplements designed to provide a combination of active ingredients for providing benefits for skincare, such as improved skin hydration, smoothness, radiance, elasticity, and reduction in fine lines and wrinkles. In some embodiments, the supplements of the disclosure combine active ingredients having a desired benefit while lacking any animal-derived components, thereby allowing consumption of the supplement by non-vegans and vegans alike. In some embodiments, the supplements of the disclosure are formulated for ingestion to provide skincare benefits. While ingestible skincare products have been developed, such products may be limited to single active ingredients, or combinations of active ingredients that lack evidence from human clinical studies. In contrast, the supplements of the present disclosure provide multiple active ingredients in a single supplement that is ingested, e.g., daily, that when combined provide clinically demonstrated benefits for skincare that are improved as compared to consuming the individual active ingredients.

In some embodiments, the supplements of the disclosure comprise an HA and a lipid extract. As further described herein, HA is a linear glycosaminoglycan that is responsible for hydration, joint lubrication, and space filling in connective tissues and organs, particularly in the skin, eyes, and joint fluid. While HA is a common component in topical ointments for improving skin condition, its benefits for skincare when consumed orally are less understood. To achieve such benefits, the disclosure provides embodiments in which the supplements comprise hyaluronic acid characterized by a particular molecular weight or range of molecular weights. In such embodiments, and without being bound by theory, the HA comprises a molecular weight that is optimal for distribution to the skin once in circulation, e.g., by providing higher distribution to skin as compared to other tissues or organs of the body such as joints. The lipid extract present in supplements of the disclosure is characterized by its combination of lipids. In some embodiments, the combination comprises a ceramide and a glycolipid. As further described herein, ceramides are the major lipid component of the intercellular spaces of the stratum corneum, the uppermost layer of the epidermis that plays a critical role in maintaining the skin's barrier function by helping to prevent moisture loss and to protect against environmental stressors. In some embodiments, supplements of the disclosure comprise an amount of a ceramide that when taken orally, contributes to a reduction in wrinkles and fine lines, promotes smooth skin, and supports skin hydration.

Further, in some embodiments, the supplements of the disclosure comprise a dosage form that combines the active ingredients in a single supplement. In some embodiments, the dosage form comprises a capsule-in-capsule format (also referred to herein as a “nested capsule” dosage form). In some embodiments, the capsule-in-capsule format comprises an outer capsule and an inner capsule, the outer capsule comprising the lipid extract in an oil and the inner capsule comprising the HA in a solid. A benefit of the capsule-in-capsule format is to maintain the active ingredients, which have distinct physical properties, separate and in a physical state customized for each. HA and a lipid extract typically do not mix well together, as the first is hydrophilic and the second is hydrophobic. The capsule-in-capsule format keeps the active ingredients in separate compartments to prevent undesirable mixing. Moreover, HA is hygroscopic and forms a gel upon contact with water. To form a shelf-stable supplement, HA is ideally maintained in a solid state and without contact to moisture. The capsule-in-capsule format retains the HA in a solid surrounded by an inner and outer capsule, which shield the solid from exposure to moisture. Altogether, the dosage format described herein provides a supplement having shelf-stability and ease of manufacture, while also allowing for precise and consistent dosing of encapsulated active ingredients.

In some embodiments, the inner and outer capsules of the capsule-in-capsule dosage format are immediate release capsules. In such embodiments, it was considered beneficial to use immediate release capsules due to the tendency of HA to form a gel upon contact with moisture which in turn has a delayed release profile. Without being bound by theory, the immediate release capsule undergoes rapid dissolution following ingestion to release HA, which in turn forms a gel upon contact with gastric fluid that is slowly dissolved as the HA passes through the gastrointestinal tract, thereby providing optimal absorption of HA.

Daily consumption of the supplements described herein throughout comprising HA and a lipid extract provided numerous benefits for skin function and appearance in both male and female adults across a range of ages. Indeed, it was shown that subjects administered the inventive supplements described herein daily for a duration of about 12 weeks experienced one or more skin properties that were improved by at least about 8 weeks as compared to prior to the administering. For example, subjects observed improved skin smoothness, skin elasticity, skin hydration/moisturization, skin radiance, and reduced wrinkles. It was further observed that one or more improvements were significant as compared to a group of placebo subjects administered a control supplement (e.g., a visually identical supplement). As one example, it was observed that subjects administered one of the inventive supplements described herein experienced skin smoothness at one or more body sites that was improved by about 2.9× in the crow's feet area at 12 weeks, compared to placebo. It was considered surprising that this improvement was observed at 12 weeks, as subjects administered a supplement comprising HA or the lipid extract, but not both, did not experience improved skin smoothness following this duration of supplementation.

Accordingly, the present disclosure provides supplements and methods of use thereof for improving one or more skin properties in a subject.

As used herein, the indefinite articles “a” and “an” and the definite article “the” are intended to include both the singular and the plural, unless the context in which they are used clearly indicates otherwise. “At least one” and “one or more” are used interchangeably to mean that the article may include one or more than one of the listed elements.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents. Unless otherwise indicated, it is to be understood that all numbers expressing quantities, ratios, and numerical properties of ingredients, reaction conditions, and so forth, used in the specification and claims are contemplated to be able to be modified in all instances by the term “about”

### Skin Supplement Compositions

In some aspects, the present disclosure provides a supplement for improving or maintaining one or more properties of skin appearance and/or skin function.

In some embodiments, the supplement comprises an active ingredient (e.g., 1, 2, 3, 4, or more active ingredient(s)). In some embodiments, the active ingredient is characterized by having a benefit for skin care. In some embodiments, the supplement comprises a capsule (e.g., 1, 2, 3, 4, or more capsules) comprising the active ingredient. In some embodiments, the supplement is formulated as a dosage form described herein (e.g., a dosage form comprising an inner capsule and an outer capsule). In some embodiments, the dosage form provides for delivery of immiscible active ingredients (e.g., oil-soluble and aqueous-soluble active ingredients) in a single supplement, thereby obviating the need for a user to consume the active ingredients in separate supplements.

### Active Ingredients

In some embodiments, the supplement comprises a single active ingredient described herein. In some embodiments, the supplement comprises more than one active ingredient described herein. In some embodiments, the supplement comprises 2, 3, 4, 5, or more active ingredients described herein. In some embodiments, the supplement comprises two active ingredients described herein.

As used herein, the terms “active,” “active agent,” and “active ingredient” are used interchangeably and refer to a compound in a composition or a dosage form described herein intended to have a desired effect (e.g., providing a benefit for skin care). The term encompasses any molecule, chemical, composition, drug, active ingredient, or biological agent for preventing, ameliorating, or treating a disease or disorder in an individual. Further, the term encompasses small molecules (e.g., compounds comprising a molecular weight less than about 1 kDa), peptides, proteins, nucleic acids, inorganic compounds or alloys of inorganic compounds, carbohydrates, lipids, and combinations thereof. In some embodiments, the active is a naturally occurring substance or a derivative thereof. In some embodiments, the active is a synthetic (i.e., chemically synthesized) substance.

In some embodiments, the active ingredient has a benefit for skin care, such as promoting or maintaining skin appearance and/or skin function. In some embodiments, the active ingredient has a benefit for skin care when consumed orally. In some embodiments, the active ingredient has a benefit for skin care when consumed in a dosing regimen (e.g., a daily dosage regimen).

In some embodiments, the active ingredient promotes or maintains skin hydration, e.g., when administered orally in a dosing regimen. In some embodiments, the active ingredient promotes or maintains skin radiance, e.g., when administered orally in a dosing regimen. In some embodiments, the active ingredient promotes or maintains skin elasticity, e.g., when administered orally in a dosing regimen. In some embodiments, the active ingredient promotes or maintains skin barrier function, e.g., when administered orally in a dosing regimen. In some embodiments, the active ingredient promotes or maintains skin moisturization, e.g., when administered orally in a dosing regimen. In some embodiments, the active ingredient decreases or prevents skin redness, e.g., when administered orally in a dosing regimen. In some embodiments, the active ingredient decreases or prevents skin flaking, e.g., when administered orally in a dosing regimen. In some embodiments, the active ingredient decreases or prevents skin roughness, e.g., when administered orally in a dosing regimen. In some embodiments, the active ingredient decreases or prevents skin wrinkles, e.g., when administered orally in a dosing regimen.

In some embodiments, the active ingredient is a glycosaminoglycan (e.g., hyaluronic acid) present in skin. In some embodiments, the active ingredient is a lipid component (e.g., a phospholipid, a glycolipid, a sphingolipid or a combination thereof) present in skin. In some embodiments, the active ingredient is a polar lipid present in skin.

In some embodiments, the active ingredient is aqueous soluble. In some embodiments, the active ingredient is oil soluble.

In some embodiments, the active ingredient is substantially or essentially free of components derived from animals so that the supplements described herein are vegan. For example, in some embodiments, the active ingredient lacks a component derived from an animal as measured according to analytical methods known in the art.

### Hyaluronic Acid

As used herein, the term “HA,” used interchangeably with “hyaluronic acid,” refers to a polysaccharide comprising a repeating unit of glucuronic acid and N-acetylglucosamine, and variants thereof (e.g., HA modified by, e.g., oxidation, reduction, sulphation, deamidation, esterification, crosslinking, and/or substitution). The term encompasses HA obtained from various sources of animal and non-animal origin. HA is also referred to as “hyaluronan” or “hyaluronate.”

HA is a glucosaminoglycan found in the connective tissues and organs, including skin, joint fluid, blood vessels, serum, and others. Due to various factors, such as aging, exposure to ultraviolet light, dryness, the content of HA in the skin becomes low. This in turn can decrease water content of the skin and contribute to wrinkles and other dermatological signs of aging. Injection of HA fillers or topical agents containing HA are used to prevent onset of dermatological aging and improve skin moisturization. The downside is that such injections are costly and associated with undesirable side effects (e.g., pain and swelling) and topical treatments have limited efficacy.

Accordingly, the present disclosure provides skin supplements comprising HA or a salt thereof for administering to a subject to provide a skin care benefit. In some embodiments, the HA comprises a molecular weight effective for gastrointestinal absorption following ingestion. In some embodiments, the HA comprises a molecular weight effective for distribution to the skin following ingestion.

In some embodiments, the HA comprises a molecular weight (e.g., an average molecular weight or a number average molecular weight) of at least about 1 kDa and up to about 3,000 kDa. In some embodiments, the HA comprises a molecular weight (e.g., an average molecular weight or a number average molecular weight) of at least about 1 kDa and up to about 2,000 kDa. In some embodiments, the HA comprises a molecular weight (e.g., an average molecular weight or a number average molecular weight) of at least about 1 kDa and up to about 1,000 kDa. In some embodiments, the HA comprises a molecular weight (e.g., an average molecular weight or a number average molecular weight) of at least about 100 kDa and up to about 1,000 kDa. In some embodiments, the HA comprises a molecular weight (e.g., an average molecular weight or a number average molecular weight) of at least about 100 kDa and up to about 800 kDa. In some embodiments, the HA comprises a molecular weight (e.g., an average molecular weight or a number average molecular weight) of at least about 200 kDa and up to about 500 kDa. In some embodiments, the HA comprises a molecular weight (e.g., an average molecular weight or a number average molecular weight) of about 300 kDa.

Methods to measure molecular weight of HA are known in the art (e.g., viscometry, conventional size exclusion chromatography (SEC), SEC with multi-angle light scattering detector (SEC-MALLS), gel electrophoresis using monodisperse HA standards). For example, in one embodiment, the molecular weight of HA is the viscosity measurement method. The specific viscosity is measured at a temperature (e.g., about 30° C.) for a number of sample solutions comprising HA at a predetermined concentration (e.g., using a capillary tube viscometer). The reduced viscosity is calculated from the specific viscosity. The reduced viscosity is plotted versus the predetermined concentration. The limiting viscosity is determined by extrapolating the sample concentration to zero. The limiting viscosity is used in the Laurent's equation to calculate the average molecular weight. See, e.g., Laurent, et al (1960) Biochemical et Biophysical Acta 42:476.

In some embodiments, skin supplement comprises a salt of HA. In some embodiments, the salt of a HA is any salt known in the art. In some embodiments, the salt of a HA described herein is a sodium salt, a potassium salt, a calcium salt, a zinc salt, a magnesium salt, or an ammonium salt.

In some embodiments, the HA is incorporated into the skin supplement as a raw material. In some embodiments, the percentage purity of the HA in the raw material is at least about 90%, about 91%, about 92%, about 93%, about 94%, about 95%, about 96%, about 97%, about 98%, or about 99%. In some embodiments, the weight percent of the HA in the skin supplement is determined as the product of the percentage purity of the HA in the raw material and the percentage of the raw material used in the supplement.

Commercially available HA ingredients suitable for use in the skin supplement compositions of the disclosure include, but are not limited to, Hyabest(S) LF-P (Kewpie), Haplex®/Haplex®Plus Food Grade Sodium Hyaluronate (Bloomage), and non-branded hyaluronic acid (e.g., distributed by NutriScience® or Maypro®).

### Lipids/Lipid Mixtures

In some embodiments, the present disclosure provides a skin supplement comprising a lipid. In some embodiments, the lipid comprises a phospholipid. In some embodiments, the lipid comprises a glycolipid. In some embodiments, the lipid comprises a sphingolipid. In some embodiments, the sphingolipid is a ceramide. In some embodiments, the sphingolipid is a glycosylceramide.

In some embodiments, the glycolipid comprises one or more monosaccharides (e.g., galactose) linked to one or more hydrophobic moieties (e.g., a fatty acyl chain) by a glycosidic linkage. In some embodiments, the glycolipid is selected from a rhamnolipid, a glucolipid, a sophorolipid, a trehalolipid, a cellobioselipid, a galactolipid, a sulfolipid, a glycosphingolipid, and a mixture thereof. In some embodiments, the glycolipid comprises digalactosyldiacylglycerol (DGDG).

In some embodiments, the ceramide comprises a fatty acid derivative and a sphingosine base. In some embodiments, the fatty acid is saturated or unsaturated. In some embodiments, the fatty acid is mono-unsaturated. In some embodiments, the fatty acid comprises a chain length of about 14 to about 26 carbons. In some embodiments, the ceramide comprises a dihydroceramide. In some embodiments, the ceramide comprises a phytoceramide. In some embodiments, the ceramide comprises a ceramide present in the epidermis (see, e.g., Merleev, et al (2022) JCI Insight, Supplemental Data p14; Suzuki, et al (2022) J Lipid Res 63:100235; Coderch, et al (2003) Am J Clin 4:107-29).

In some embodiments, the skin supplement comprises a mixture of two or more lipids. In some embodiments, the mixture comprises a phospholipid and a glycolipid. In some embodiments, the mixture comprises a phospholipid and a sphingolipid. In some embodiments, the mixture comprises a glycolipid and a sphingolipid. In some embodiments, the mixture comprises a phospholipid, a sphingolipid, and a glycolipid. In some embodiments, the mixture comprises a ceramide and a glycolipid (e.g., DGDG). In some embodiments, the mixture comprises a glycoceramide and a glycolipid (e.g., DGDG).

In some embodiments, the skin supplement comprises a lipid extract. In some embodiments, the lipid extract is obtained from a plant material. In some embodiments, the lipid extract is obtained from algae. In some embodiments, the lipid extract is obtained from any plant material known in the art to have high ceramide content (e.g., a cereal, an oleaginous plant (e.g., soybean), a tuber, a root vegetable (e.g., potato, sweet potato, yam), or spinach). In some embodiments, the plant material is a cereal. In some embodiments, the cereal flour is obtained from a grain. In some embodiments, the grain is selected from wheat, wheat gluten, rice, millet, oats, barley, maize, and sorghum.

Methods to obtain lipid extracts from cereal flour are known in the art (see, e.g., FR Appln No. 9904915). In some embodiments, the method comprises contacting (e.g., stirring) the cereal flour with a solvent or mixture of solvents for a duration of one or more hours, such as at ambient temperature or higher (e.g., about 20° C. to about 60° C.). In some embodiments, the method comprises obtaining a filtrate comprising polar and/or neutral lipids. In some embodiments, the method comprises concentrating the filtrate (e.g., under vacuum) to obtain an oil. In some embodiments, the method comprises processing the oil (e.g., via dehydration and/or filtration) to provide a liquid comprising polar and/or neutral lipids. In some embodiments, the method comprises extracting polar lipids from the liquid, such as by contacting the liquid with an organic solvent to induce precipitation of the polar lipids. In some embodiments, the precipitate is collected to provide a composition of polar lipids. In some embodiments, the organic solvent is concentrated (e.g., under vacuum) to provide a composition of neutral lipids.

In some embodiments, the lipid extract is obtained from wheat, e.g., according to a method described herein. In some embodiments, the lipid extract is obtained from Triticum vulgare/aestivum.

In some embodiments, the lipid extract comprises a weight percent of a glycolipid (e.g., DGDG), wherein the weight percent is at least about 15%. In some embodiments, the weight percent is about 15%. In some embodiments, the lipid extract comprises a weight percent of a ceramide or a glycosylceramide, wherein the weight percent is at least about 2%. In some embodiments, the weight percent is about 2%. In some embodiments, the lipid extract comprises a weight percent of a glycolipid (e.g., DGDG) of at least about 15% and a weight percent of a ceramide or a glycosylceramide of at least about 2%. In some embodiments, the lipid extract comprises a weight percent of a glycolipid (e.g., DGDG) of about 15% and a weight percent of a ceramide or a glycosylceramide of about 2%.

Commercially available lipid extracts suitable for use in the skin supplement compositions of the disclosure include, but are not limited to, Ceratiq® (PLT Health Solutions), Ceramosides™ (Seppic), CeraLOK® (Anderson Advanced Ingredients), or Konjac ceramide (Daicel Corporation).

### Excipients

In some embodiments, the skin supplement composition comprises an excipient (e.g., 1, 2, 3, 4, 5, or more excipients). In some embodiments, the excipient comprises a binder, a lubricant, a flow aid, a colorant, a rate-controlling polymer, a lubricant, a disintegrant, a glidant, viscosity adjuster, a filler, a diluent, or a combination thereof.

In some embodiments, the excipient comprises a binder. A “binder” refers to a substance added to a tableting mixture to improve cohesion and plasticity, which in turn enhances processability when formulated into a tablet. In some embodiments, the binder is a natural polymer, e.g., a natural polymer selected from Arabic gum, gelatin, sodium alginate, pullulan, starch, pregelatinized starch, and tragant. In some embodiments, the binder is a semi-synthetic polymer, e.g., a semi-synthetic polymer selected from Carboxymethylcellulose Sodium, Dextrin, Hydroxyethylcellulose, Hydroxypropylcellulose (HPC), Hypromellose, Maltodextrin, and Methylcellulose. In some embodiments, the binder is a synthetic polymer, e.g., a synthetic polymer selected from copovidone, a macrogols, a polyvinyl alcohols (PVA), and povidone. In some embodiments, the binder is selected from a Fatty alcohols, a Fat, a Wax, a Hydrated Rizinius Oil, and Stearic Acid. In some embodiments, the binder is selected from Povidone (Polyvinylpyrrolidone, PVP), HPC, Microcrystalline cellulose (MCC), Polyethylene glycol (PEG), Gelatin, Starch, a Carbomers and Sodium carboxymethyl cellulose (NaCMC). In some embodiments, the binder is MCC.

In some embodiments, the excipient comprises a lubricant. A “lubricant,” “flow aid” or a “glidant” each refer to a substance added to a tableting mixture for the purpose of improving flow properties, reducing interparticle friction, and reducing sticking to machinery used in tablet production. In some embodiments, the lubricant is selected from Magnesium stearate, Stearic acid, Calcium stearate, Sodium stearyl fumarate, Polyethylene glycols, Silicone dioxide, Talc, Beeswax, and Hydrogenated vegetable oil. In some embodiments, the lubricant comprises silicon dioxide. In some embodiments, the lubricant comprises ascorbyl palmitate powder. In some embodiments, the glidant or the flow aid comprises calcium palmitate, magnesium stearate, fumed silica, starch, talc, or a combination thereof. In some embodiments, the glidant or flow aid comprises silica.

In some embodiments, the lubricant comprises an oil. In some embodiments, the oil is liquid above about −110° F., above about −100° F., above about −90° F., above about −80° F., above about −70° F., above about −60° F., above about −50° F., −40° F., above about −30° F., above about −20° F., above, about −10° F., above about 0° F., above about 10° F., above about 20° F., above about 30° F., above about 40° F., above about 50° F., above about 60° F., above about 70° F., above about 80° F., or above about 90° F. In some embodiments, the lubricant comprises a hydrogenated vegetable oil. In some embodiments, the lubricant comprises sunflower oil. In some embodiments, the oil is a medium chain triglyceride (MCT). In some embodiments, the lubricant comprises MCT oil. The MCT may be from any suitable source. In some embodiments, the MCT is a triglyceride having aliphatic tails containing six, seven, eight, nine, ten, eleven, or twelve carbons. In some embodiments, the MCT is derived from a natural source, such as coconut oil. In some embodiments, the oil is a coconut MCT oil. In some embodiments, the oil is a sunflower oil. In some embodiments, the lubricant comprises a tocopherol.

In some embodiments, the excipient comprises a disintegrant. A “disintegrant” refers to a substance added to a tableting mixture for the purpose of facilitating disintegration and dissolution into smaller particles that dissolve more rapidly into the gastrointestinal fluid following enteral administration (see USP 41-NF 36 General Chapter <701> Disintegration). In some embodiments, the disintegrant is selected from alginic acid, bentonite, MCC, powdered cellulose, guargalactomannan, and a combination thereof. In some embodiments, the disintegrant comprises a starch (e.g., corn starch, potato starch, pregelatinized starch, sodium starch glycolate, and/or starch 1500). In some embodiments, the starch is corn starch, rice starch, pea starch, tapioca starch, or a combination thereof. In some embodiments, the disintegrant comprises a cellulose or a derivative thereof (e.g., microcrystalline cellulose, croscarmellose sodium, sodium carboxymethyl cellulose, and hydroxypropyl methylcellulose). In some embodiments, the disintegrant comprises a gum (e.g., guar gum, xanthan gum, and locust bean gum). In some embodiments, the disintegrant comprises a calcium silicate (e.g., dicalcium phosphate or tricalcium phosphate). In some embodiments, the disintegrant comprises sodium alginate, cross-linked polyvinylpyrrolidone, and/or chitosan. In some embodiments, the disintegrant is selected from Carboxymethylcellulose Calcium, Carmellose Sodium, Croscarmellose Sodium, Sodium Starch Glycolate Type A or Type B, Low-Substituted Carboxymethylcellulose Sodium, Low-Substituted Hydroxypropyl Cellulose, and crospovidon. In some embodiments, the disintegrant comprises psyllium husk, oat fiber, and a combination thereof.

In some embodiments, the excipient comprises a flavor agent or odorant agent, or a combination thereof. A flavor agent may comprise one or more compounds that impart a flavor to the dietary supplement composition. An odorant agent comprises one or more compounds that impart an odor to the dietary supplement composition. In some embodiments, a flavor agent may also act as an odor agent. Including one or more flavor agents and/or odorant agents in the oil may increase compliance with a dosing regimen. The flavor agent or odorant agent may be derived from natural sources. In some embodiments, the odorant agent is a plant oil, a compound derived from plants, or an oil comprising one or more compounds derived from a plant. In some embodiments, the flavor agent is a plant oil, a compound derived from plants, or an oil comprising one or more compounds derived from a plant. For example, in certain embodiments, the flavor agent or odorant agent comprises a flavor, oil, extract, or scent selected from the group consisting of mint, vanilla, ginger, grapefruit, rosemary, lemon, lime, and orange. For example, the flavor agent or odorant agent may be vanilla flavor, mint flavor, mint oil, ginger flavor, ginger oil, grapefruit flavor, grapefruit oil, rosemary flavor, rosemary oil, lemon flavor, lemon oil, orange flavor, or orange oil. In one embodiment, the flavor agent or odorant agent comprises a mint oil. In one example, the mint oil is peppermint oil. In another embodiment, the flavor agent or odorant agent comprises vanilla flavor. In one embodiment, the vanilla flavor is derived from natural sources.

In some embodiments, the excipient comprises a colorant. A “colorant” refers to a substance added to a tableting mixture for the purpose of providing color. In some embodiments, the colorant is selected from any known in the art (see, e.g., Biswal, et al (2015) IJPCBS 5:1004). In some embodiments, the colorant is naturally derived. In some embodiments, the colorant is selected from a Polyphenol derivative, an isoprene derivative, a Tetrapyrrole derivative, a Ketone derivative, a Quinone derivative, and a combination thereof. In some embodiments, the colorant comprises a carotenoid (e.g., capsanthin, crocin, lutein, bixin), an indol derivative (e.g., indigotin, bromoindigotin), a oxyindol glycoside (e.g., betanin), a diaryl heptanoid (e.g., curcumin), a benzopyrone (e.g., haematin). In some embodiments, the colorant comprises beta-carotene. In some embodiments, the colorant comprises indigotine. In some embodiments, the colorant comprises spirulina extract. In some embodiments, the colorant comprises saffron extract. In some embodiments, the colorant comprises riboflavin. In some embodiments, the colorant comprises turmeric. In some embodiments, the colorant comprises purple carrot.

### Supplement Dosage Form

The present disclosure provides dosage forms comprising the skin supplement compositions described herein. In some embodiments, the dosage form is any solid dosage form known in the art. As used herein, the term “solid dosage form” refers to a dosage form that is a solid at ambient conditions. In some embodiments, the solid dosage form comprises a gummy, a tablet, a capsule, or a powder. In some embodiments, the skin supplement composition is formulated in the dosage form according to any method known in the art or described herein.

In some embodiments, the composition comprises a dose of an active ingredient described herein, wherein the composition is formulated in a dosage form described herein. In some embodiments, the dosage form comprises a single dose of an active ingredient described herein. In some embodiments, the dosage form comprises more than one dose of an active ingredient described herein. In some embodiments, the dosage form comprises an excipient. In some embodiments, the dosage form comprises more than one excipient.

In some embodiments, the dosage form comprises an HA described herein. In some embodiments, the dosage form comprises a mixture of HAs described herein. In some embodiments, the dosage form comprises a dose of the HA, wherein the dose is at least about 100 mg, about 110 mg, about 120 mg, about 130 mg, or about 140 mg, about 150 mg, about 160 mg, about 170 mg, about 180 mg, about 190 mg, about 200 mg, about 210 mg, about 220 mg, about 230 mg, or about 240 mg. In some embodiments, the dosage form comprises the HA in a weight percent of the total weight of at least about 5%, about 10%, about 15%, about 20%, or about 25%. In some embodiments, the weight percent is about 5% to about 30%, about 10% to about 25%, or about 15% to about 25%. In some embodiments, the weight percent is about 15% to about 25%. In some embodiments, the weight percent is about 20% to about 25%.

In some embodiments, the dosage form comprises a lipid or a lipid extract described herein. In some embodiments, the dosage form comprises a dose of the lipid or the lipid extract, wherein the dose is at least about 300 mg to about 400 mg, or about 310 mg, about 320 mg, about 330 mg, about 340 mg, about 350 mg, about 360 mg, about 370 mg, about 380 mg, or about 390 mg. In some embodiments, the dosage form comprises the lipid or the lipid extract in a weight percent of the total weight of at least about 5%, about 10%, about 15%, about 20%, about 25%, about 30%, about 35%, about 40%, about 45%, about 50%, about 55% or about 60%. In some embodiments, the weight percent is about 20% to about 60%, about 25% to about 55%, about 30% to about 50%, about 35% to about 50%, or about 45% to about 50%. In some embodiments, the weight percent is about 45% to about 55%.

In some embodiments, the composition comprises a HA described herein and a lipid described herein. In some embodiments, the composition comprises a HA described herein and a lipid extract described herein.

In some embodiments, the dosage form comprises an excipient (e.g., 1, 2, 3, 4, 5 or more excipients) described herein. In some embodiments, the dosage form comprises a lubricant, a binder, a disintegrant, a colorant, an odorant, a flavor agent, a rate controlling polymer, or a combination thereof. In some embodiments, the dosage form comprises the excipient in a weight percent of the total weight of less than about 80%, about 75%, about 70%, about 65%, about 60%, about 55%, about 50%, about 45%, about 40%, about 35%, about 30%, about 25%, about 20%, about 15%, or about 10%. In some embodiments, the weight percent is about 10% to about 50%, about 15% to about 40%, or about 20% to about 35%.

In some embodiments, the dosage form comprises a capsule comprising the skin supplement composition. As used herein, the term “capsule” includes instant release capsules, sustained release capsules, coated instant release capsules, coated sustained release capsules, delayed release capsules and coated delayed release capsules.

In some embodiments, the capsule is an immediate release capsule. In some embodiments, the immediate release capsule is formulated to release its contents in less than about 1 hour, about 50 minutes, about 40 minutes, about 30 minutes, about 20 minutes, or about 10 minutes after ingestion. In some embodiments, the capsule is an extended release capsule. In some embodiments, the extended release capsule is formulated to release its contents after at least 10, 20, 30, 40, 50, 60, 70, 80, 90, or 100 or more minutes after ingestion. In some embodiments, the extended release capsule is configured to release its contents after entering the small intestine of the subject. In some embodiments, the extended release capsule is configured to bypass the stomach of the subject and release its contents after entering the intestine of the subject.

In some embodiments, the capsule is size 0. In some embodiments, the capsule is elongated size 0. In some embodiments, the capsule is size 00. In some embodiments, the capsule is elongated size 00. In some embodiments, the capsule is size 1. In some embodiments, the capsule is size 2. In some embodiments, the capsule is size 3. In some embodiments, the capsule is round, oval, oblong, or nonstandard in shape. In some embodiments, the capsule comprises a minimum fill volume of about 0.01 mL to about 0.2 mL. In some embodiments, the capsule comprises a maximum fill volume of about 0.05 mL to about 0.3 mL.

In some embodiments, the capsule is translucent. In some embodiments, the capsule is semi-opaque. In some embodiments, the capsule is opaque.

In some embodiments, the shell of the capsule comprises a polymer (e.g., 1, 2, 3, 4 or more polymers). In some embodiments, the polymer comprises hydroxypropyl methylcellulose (hypromellose), gellan gum, pullalan, or any combinations thereof. In some embodiments, the capsule comprises an excipient, such as one or more binders, fillers, diluents, lubricants, or any combinations thereof. In some embodiments, the capsule comprises hypromellose, gellan gum, and silica. In some embodiments, the capsule consists essentially of hypromellose, gellan gum, and silica.

In some embodiments, the capsule is essentially free of ingredients derived from animals. For example, the one or more capsules may comprise less than 1 wt %, less than 0.1 wt %, or less than 0.01 wt % gelatin. In some embodiments, the capsule is essentially free of gelatin. In some embodiments, the capsule is vegetarian.

In some embodiments, the shell of the capsule is coated. In some embodiments, the shell comprises an enteric or delayed release coating, a sustained release coating, a barrier coating, or a combination thereof.

In some embodiments, the dosage form comprises more than one capsule comprising the skin supplement composition. In some embodiments, the dosage form comprises two capsules comprising the skin supplement composition.

In some embodiments, the dosage form comprises an inner capsule and an outer capsule, wherein the inner capsule is contained in the outer capsule. In some embodiments, the inner capsule comprises an active ingredient of the skin supplement composition. In some embodiments, the outer capsule comprises an active ingredient of the skin supplement composition. In some embodiments, both the inner capsule and the outer capsule comprises an active ingredient of the skin supplement composition.

In some embodiments, the outer capsule comprises an active ingredient in liquid form. In some embodiments, the outer capsule comprises an active ingredient in solid form. In some embodiments, the inner capsule comprises an active ingredient in liquid form. In some embodiments, the liquid form is an oil. In some embodiments, the solid form is a powder.

In some embodiments, the outer capsule comprises a liquid fill phase, wherein the liquid fill phase is lipophilic. In some embodiments, the liquid fill phase comprises an oil carrying an active ingredient described herein (e.g., a lipid extract described herein). In some embodiments, the oil comprises an omega-3 fatty acid, a vegetable oil, a mineral oil, a tocopherol, a food grade oil, or a combination thereof. In some embodiments, the vegetable oil is castor bean oil, coconut oil, peanut oil, palm kernel oil, canola oil, avocado oil, evening primrose oil, rice bran oil, borage oil, sunflower oil, soybean oil, palm oil, corn oil, olive oil, pumpkin seed oil, grape seed oil, sesame oil, argan oil and safflower oil. In some embodiments, the active ingredient is present as a suspension, an emulsion, a solution, or a micro-emulsion in the oil.

In some embodiments, the inner capsule comprises an active ingredient in a solid. In some embodiments, the outer capsule comprises an active ingredient in an oil. In some embodiments, the outer capsule comprises the inner capsule surrounded by the oil.

In some embodiments, the inner capsule is an immediate release capsule or an extended release capsule. In some embodiments, the outer capsule is an immediate release capsule or an extended release capsule. In some embodiments, the inner capsule and the outer capsule are both immediate release capsules.

In some embodiments, the total internal volume of the inner capsule is less than the total internal volume of the outer capsule.

In some embodiments, the outer capsule is a size 00 and the inner capsule is a size 2 or 3.

In some embodiments, the dosage forms described herein are produced using any suitable methods known in the art. In some embodiments, the method comprises introducing a composition described herein (e.g., a composition comprising an active ingredient and an excipient) into a cavity present in a capsule described herein. In some embodiments, the method further comprises sealing the capsule. In some embodiments, the composition is introduced into the cavity as a liquid fill phase. In some embodiments, the composition is introduced into the cavity as a solid fill phase.

In some embodiments, the method comprises introducing a composition described herein (e.g., a composition comprising an active ingredient and an excipient) into a cavity present in a first capsule and introducing the first capsule into a cavity present in a second capsule. In some embodiments, the method further comprises filling the empty volume of the cavity present in the second capsule with a composition described herein (e.g., a composition comprising an active ingredient and an excipient). In some embodiments, the method further comprises sealing the second capsule.

### Exemplary Dosage Forms

In some embodiments, the present disclosure provides a dosage form comprising an inner capsule and an outer capsule, wherein the inner capsule comprises an HA described herein, wherein the outer capsule comprises a lipid extract described herein, and wherein the inner capsule is contained in the outer capsule.

In some embodiments, the present disclosure provides a dosage form comprising an inner capsule and an outer capsule, wherein the inner capsule comprises HA having a molecular weight (e.g., an average molecular weight or a number average molecular weight) effective for gastrointestinal absorption following ingestion, wherein the outer capsule comprises a lipid extract, and wherein the inner capsule is contained in the outer capsule.

In some embodiments, the present disclosure provides a dosage form comprising an inner capsule and an outer capsule, wherein the inner capsule comprises HA having a molecular weight (e.g., an average molecular weight or a number average molecular weight) effective for gastrointestinal absorption and distribution to skin following ingestion, wherein the outer capsule comprises a lipid extract, and wherein the inner capsule is contained in the outer capsule.

In some embodiments, the present disclosure provides a dosage form comprising an inner capsule and an outer capsule, wherein the inner capsule comprises HA having a molecular weight (e.g., an average molecular weight or a number average molecular weight) of at least about 200 kDa and up to about 500 kDa, wherein the outer capsule comprises a lipid extract, and wherein the inner capsule is contained in the outer capsule.

In some embodiments, the present disclosure provides a dosage form comprising an inner capsule and an outer capsule, wherein the inner capsule comprises HA having a molecular weight (e.g., an average molecular weight or a number average molecular weight) of at least about 300 kDa, wherein the outer capsule comprises a lipid extract, and wherein the inner capsule is contained in the outer capsule.

In some embodiments, the present disclosure provides a dosage form comprising an inner capsule and an outer capsule, wherein the inner capsule comprises HA having a molecular weight (e.g., an average molecular weight or a number average molecular weight) effective for gastrointestinal absorption following ingestion, wherein the outer capsule comprises a lipid extract, wherein the lipid extract comprises a ceramide and a glycolipid, and wherein the inner capsule is contained in the outer capsule.

In some embodiments, the present disclosure provides a dosage form comprising an inner capsule and an outer capsule, wherein the inner capsule comprises HA having a molecular weight (e.g., an average molecular weight or a number average molecular weight) effective for gastrointestinal absorption and distribution to skin following ingestion, wherein the outer capsule comprises a lipid extract, wherein the lipid extract comprises a ceramide and a glycolipid, and wherein the inner capsule is contained in the outer capsule.

In some embodiments, the present disclosure provides a dosage form comprising an inner capsule and an outer capsule, wherein the inner capsule comprises HA having a molecular weight (e.g., an average molecular weight or a number average molecular weight) effective for gastrointestinal absorption following ingestion, wherein the outer capsule comprises a lipid extract, wherein the lipid extract comprises a ceramide in a weight percent of at least about 2% and a glycolipid in a weight percent of at least about 15%, and wherein the inner capsule is contained in the outer capsule.

In some embodiments, the present disclosure provides a dosage form comprising an inner capsule and an outer capsule, wherein the inner capsule comprises HA having a molecular weight (e.g., an average molecular weight or a number average molecular weight) of at least about 200 kDa and up to about 500 kDa, wherein the outer capsule comprises a lipid extract, wherein the lipid extract comprises a ceramide in a weight percent of at least about 2% and a glycolipid in a weight percent of at least about 15%, and wherein the inner capsule is contained in the outer capsule.

In some embodiments, the present disclosure provides a dosage form comprising an inner capsule and an outer capsule, wherein the inner capsule comprises HA having a molecular weight (e.g., an average molecular weight or a number average molecular weight) of at least about 300 kDa, wherein the outer capsule comprises a lipid extract, wherein the lipid extract comprises a ceramide in a weight percent of at least about 2% and a glycolipid in a weight percent of at least about 15%, and wherein the inner capsule is contained in the outer capsule.

In some embodiments, the inner capsule comprises the HA in a solid. In some embodiments, the outer capsule comprises the lipid extract in an oil.

In some embodiments, the inner capsule comprises a partial dose of the HA. In some embodiments, the inner capsule comprises a dose of the HA. In some embodiments, the inner capsule comprises more than one dose of the HA. In some embodiments, the dose of HA is about 100 mg, about 110 mg, about 120 mg, about 130 mg, or about 140 mg, about 150 mg, about 160 mg, about 170 mg, about 180 mg, about 190 mg, about 200 mg, about 210 mg, about 220 mg, about 230 mg, or about 240 mg.

In some embodiments, the inner capsule comprises an amount of the HA, wherein the amount is a weight percent of the total dosage form, wherein the weight percent is about 10%, about 15%, about 20%, about 25%, or about 30%. In some embodiments, the weight percent is about 15% to about 30%, about 15% to about 25%, or about 20% to about 30%. In some embodiments, the weight percent is about 20%, 21%, 22%, 23%, 24%, or 25%.

In some embodiments, the outer capsule comprises a partial dose of the lipid extract. In some embodiments, the outer capsule comprises a dose of the lipid extract. In some embodiments, the outer capsule comprises more than one dose of the lipid extract. In some embodiments, the dose of the lipid extract is about 70 mg to about 400 mg. In some embodiments, the dose of lipid extract is about 300 mg to about 400 mg, or about 310 mg, about 320 mg, about 330 mg, about 340 mg, about 350 mg, about 360 mg, about 370 mg, about 380 mg, or about 390 mg. In some embodiments, the dose of the lipid extract is about 350 mg.

In some embodiments, the outer capsule comprises an amount of the lipid extract, wherein the amount is a weight percent of the total dosage form, wherein the weight percent is about 30%, about 35%, about 40%, about 45%, about 50%, or about 55%. In some embodiments, the weight percent is about 30% to about 55%, about 40% to about 50%, or about 45% to about 55%.

In some embodiments, the lipid extract is from a plant material. In some embodiments, the lipid extract is from a cereal flour. In some embodiments, the lipid extract is from a tuber. In some embodiments, the lipid extract is from a root vegetable. In some embodiments, the lipid extract is from a grain. In some embodiments, the grain is selected from wheat, wheat gluten, rice, millet, oats, barley, maize, and sorghum. In some embodiments, the lipid extract is from wheat (e.g., Triticum vulgare/aestivum).

In some embodiments, the inner capsule further comprises an excipient described herein (e.g., 1, 2, 3, 4, 5, or more excipients). In some embodiments, the inner capsule further comprises MMC, ascorbyl palmitate, silicon dioxide, and sunflower oil.

In some embodiments, the outer capsule further comprises an excipient described herein (e.g., 1, 2, 3, 4, 5, or more excipients). In some embodiments, the outer capsule further comprises mixed tocopherols.

In some embodiments, the present disclosure provides a dosage form comprising an inner capsule and an outer capsule, wherein the inner capsule comprises an HA in a dose of about 100 mg to about 240 mg in a solid, wherein the HA comprises a molecular weight (e.g., an average molecular weight or a number average molecular weight) of about 200 kDa to about 500 kDa, wherein the outer capsule comprises a wheat lipid extract in a dose of about 70 mg to about 400 mg in an oil, and wherein the inner capsule is contained in the outer capsule.

In some embodiments, the present disclosure provides a dosage form comprising an inner capsule and an outer capsule, wherein the inner capsule comprises an HA in a dose of about 100 mg to about 240 mg in a solid, wherein the HA comprises a molecular weight (e.g., an average molecular weight or a number average molecular weight) of about 300 kDa, wherein the outer capsule comprises a wheat lipid extract in a dose of about 70 mg to about 400 mg in an oil, and wherein the inner capsule is contained in the outer capsule.

In some embodiments, the present disclosure provides a dosage form comprising an inner capsule and an outer capsule, wherein the inner capsule comprises an HA in a dose of about 120 mg in a solid, wherein the HA comprises a molecular weight (e.g., an average molecular weight or a number average molecular weight) of about 200 kDa to about 500 kDa, wherein the outer capsule comprises a wheat lipid extract in a dose of about 350 mg in an oil, and wherein the inner capsule is contained in the outer capsule.

In some embodiments, the present disclosure provides a dosage form comprising an inner capsule and an outer capsule, wherein the inner capsule comprises an HA in a dose of about 120 mg in a solid, wherein the HA comprises a molecular weight (e.g., an average molecular weight or a number average molecular weight) of about 300 kDa, wherein the outer capsule comprises a wheat lipid extract in a dose of about 350 mg in an oil, and wherein the inner capsule is contained in the outer capsule.

In some embodiments, the oil is mixed tocopherols. In some embodiments, the inner capsule further comprises MCC, ascorbyl palmitate, silicon dioxide, and sunflower oil.

In some embodiments, the inner capsule is an immediate release capsule. In some embodiments, the outer capsule is an immediate release capsule.

In some embodiments, the outer capsule is size 0. In some embodiments, the outer capsule is elongated size 0. In some embodiments, the outer capsule is size 00. In some embodiments, the outer capsule is elongated size 00. In some embodiments, the inner capsule is size 2. In some embodiments, the inner capsule is size 3.

In some embodiments, the outer capsule is a size 00 and the inner capsule is a size 2.

In some embodiments, the inner capsule is translucent. In some embodiments, the inner capsule is semi-opaque. In some embodiments, the outer capsule is translucent. In some embodiments, the outer capsule is semi-opaque.

### Methods of Use

In some embodiments, the disclosure provides methods for providing an improved skin property of a subject, comprising administering to the subject a skin supplement composition or dosage form thereof described herein.

In some embodiments, the subject is administered the skin supplement composition or dosage form thereof in a dosing regimen. In some embodiments, the dosing regimen comprises once per day. In some embodiments, the dosing regimen comprises twice per day. In some embodiments, the dosing regimen comprises a duration of at least about 4 weeks, about 5 weeks, about 6 weeks, about 7 weeks, about 8 weeks, about 9 weeks, about 10 weeks, about 11 weeks, or about 12 weeks.

In some embodiments, the skin property is improved as compared to prior to the dosing regimen.

In some embodiments, the dosing regimen comprises a duration of at least about 12 weeks, wherein the skin property is improved by at least about 6 weeks as compared to prior to the dosing regimen. In some embodiments, the skin property is improved by at least about 7 weeks as compared to prior to the dosing regimen. In some embodiments, the skin property is improved by at least about 8 weeks as compared to prior to the dosing regimen. In some embodiments, the skin property is improved by at least about 9 weeks as compared to prior to the dosing regimen. In some embodiments, the skin property is improved by at least about 10 weeks as compared to prior to the dosing regimen. In some embodiments, the skin property is improved by at least about 11 weeks as compared to prior to the dosing regimen. In some embodiments, the skin property is improved by at least about 12 weeks as compared to prior to the dosing regimen.

In some embodiments, the skin property is improved at a site of the body of the subject. In some embodiments, the site is present on the face, a leg, an arm, the torso, the neck, a hand, a foot, or a combination thereof.

In some embodiments, the disclosure provides a method for providing an improved skin property in a plurality of subjects, comprising administering to each subject a skin supplement composition or dosage form thereof described herein. In some embodiments, each subject is administered the composition or dosage form thereof in a dosing regimen. In some embodiments, the dosing regimen comprises administering the composition or dosage form thereof once per day for at least about 4 weeks, about 5 weeks, about 6 weeks, about 7 weeks, about 8 weeks, about 9 weeks, about 10 weeks, about 11 weeks, or about 12 weeks. In some embodiments, the dosing regimen comprises administering the composition or dosage form thereof for a duration of at least about 12 weeks, wherein the skin property is improved in a proportion of the subjects of the plurality by at least about 6 weeks, about 7 weeks, about 8 weeks, about 9 weeks, about 10 weeks, or about 12 weeks as compared to prior to the dosing regimen. In some embodiments, the proportion of the subjects that experience an improved skin property is compared to a control group, wherein subjects of the control group are administered a placebo. In some embodiments, the proportion of subjects that experience an improved skin property is greater compared to the control group.

In some embodiments, the improved skin property comprises reduced wrinkles. As used herein, the term “skin wrinkle,” “skin wrinkles,” “wrinkle,” or “wrinkles” each refer to lines, folds, ridges, and/or creases in the skin resulting from aging or exposure to environmental conditions (e.g., ultraviolet light). In some embodiments, a wrinkle is a coarse line (e.g., a line having a depth of greater than about 1 mm). In some embodiments, a wrinkle is a fine line (e.g., a line having a depth of less than about 1 mm). Methods for measuring wrinkles are known in the art. In some embodiments, the method comprises an objective assessment, such as, but not limited to, image analysis (see, e.g., Hamer et al (2015) Skin Res Technol 21:392), 3D fringe projection, Antera 3D (see, e.g., Anqi, et al (2022) Skin Res Technol 28:840), Stephens Wrinkle Analysis Raking Light (SWIRL) (see, e.g., Li, et al (2013) Skin Res Technol 19:492), silicon replicas, Visioscan VC98 (Courage & Khazaka), and/or VISIA Skin analysis (Canfield Scientific). In some embodiments, the method comprises a subjective assessment, such as, but not limited to Stephens Wrinkle Analysis Raking Light (AFLS), 4-point facial wrinkle scale, 5-point investigator's global assessment (IGA), self-evaluation, dermatological evaluation, Lemperle score (see, e.g., Lemperle, et al (2001) Plast Reconstr Surg 108:1735), and/or Modified Fitzpatrick Wrinkle scale (MFWS).

In some embodiments, the improved skin property comprises increased skin smoothness. As used herein, the term “skin smoothness” refers to evenness of skin texture. Methods for measuring skin smoothness are known in the art. In some embodiments, the method comprises an objective assessment, such as, but not limited to, image analysis, 3D fringe projection, PRIMOS (see, e.g., Bloemen, et al (2011) J Am Acad Dermatol 64:706), Antera 3D, optical coherence tomography (OCT), DermaTop, VISIA-CR, and/or Visioscan VC98 (Courage & Khazaka). In some embodiments, the method comprises a subjective assessment, such as, but not limited to, IGA (see, e.g., Birnbaum, et al (2017) J Cosmet Dermatol 16:120), ASRS (see, e.g., Jain, et al (2017) J Cosmet Dermatol 16:132), and/or Tactile Roughness Scale (see, e.g., Fabi, et al (2017) J Clin Aesthet Dermatol 10:14).

In some embodiments, the improved skin property comprises increased skin hydration. As used herein, the term “skin hydration” refers to the moisture content of the skin. Methods for measuring skin hydration are known in the art. In some embodiments, the method comprises an objective assessment, such as, but not limited to, transepidermal water loss (TEWL), Corneometer, aquaflex, epsilon, MoistureMeter D, and/or Tewameter. In some embodiments, the method comprises an subjective assessment, such as, but not limited to, IGA (see, e.g., Birnbaum, et al (2017) J Cosmet Dermatol 16:120), and/or the 10-point Dryness Scale (see, e.g., Werschler, et al (2011) J Clin Aesthet Dermatol 4:51).

In some embodiments, the improved skin property comprises increased skin elasticity. As used herein, the term “skin elasticity” refers to the ability of the skin to recoil with manipulation. Methods to measure skin elasticity are known in the art. In some embodiments, the method comprises an objective assessment, such as, but not limited to, Cutometer, torquemeter (see, e.g., Boyce, et al (2000) J. Burn Care Rehabil 21:55), indentation, angular rotation techniques, and/or Ballistometer. In some embodiments, the method comprises a subjective assessment, such as, but not limited to, facial laxity rating scale (FLR) (see, e.g., Silva (2016) Dermatol Surg 42:1370).

In some embodiments, the improved skin property comprises increased skin radiance. As used herein, the term “skin radiance” refers to the ability of skin to glow or reflect light. Methods to measure skin radiance are known in the art. In some embodiments, the method comprises an objective assessment, such as, but not limited to, optical methods (see, e.g., Petitjean, et al (2007) Skin Res Technol 13:2), glossymeter, and/or dermaspectrophotometer. In some embodiments, the method comprises a subjective assessment, such as, but not limited to, C.L.B.T. score (see, e.g., Musnier, et al (2004) Skin Res Technol 10:50) and/or 10-point Skin Tone Evenness Scale (Makino, et al (2017) J Drugs Dermatol 16:36).

In some embodiments, the subject is male or female. In some embodiments, the subject is an adult. In some embodiments, the subject is 18 years or older. In some embodiments, the subject is about 18 to about 36, about 24 to about 48, about 32 to about 56, about 36 to about 64, or about 40 to about 72 years old.

### Exemplary Methods

In some embodiments, the disclosure provides a method for improving a skin property (e.g., skin smoothness, skin elasticity, skin hydration, skin radiance, and/or a reduction in fine lines and wrinkles) at a site of the body of a subject, comprising administering to the subject a dosage form described herein in a dosing regimen, wherein the dosage form comprises an inner capsule and an outer capsule, wherein the inner capsule comprises an HA described herein, the outer capsule comprises a lipid extract described herein, and wherein the inner capsule is contained in the outer capsule. In some embodiments, the site of the body is on the face, arm, leg, torso, hand, foot, or a combination thereof. In some embodiments, the skin property (e.g., skin smoothness, skin elasticity, skin hydration, skin radiance, and/or a reduction in fine lines and wrinkles) is improved on the face. In some embodiments, the skin property (e.g., skin smoothness, skin elasticity, skin hydration, skin radiance, and/or a reduction in fine lines and wrinkles) is improved on the thigh. In some embodiments, the skin property (e.g., skin smoothness, skin elasticity, skin hydration, skin radiance, and/or a reduction in fine line and wrinkles) is improved on the arm. In some embodiments, the skin property (e.g., skin smoothness, skin elasticity, skin hydration, skin radiance, and/or a reduction in fine lines and wrinkles) is improved on the torso.

In some embodiments, the skin property (e.g., skin smoothness, skin elasticity, skin hydration, skin radiance, and/or a reduction in fine lines and wrinkles) of the subject is improved as compared to prior to the dosing regimen. In some embodiments, the dosing regimen comprises administering the dosage form at least once daily for a duration of at least about 12 weeks, wherein the skin property (e.g., skin smoothness, skin elasticity, skin hydration, skin radiance, and/or a reduction in fine lines and wrinkles) is improved by at least about 6 weeks, about 7 weeks, about 8 weeks, about 9 weeks, about 10 weeks, about 11 weeks, or about 12 weeks as compared to prior to the dosing regimen. In some embodiments, the skin property (e.g., skin smoothness, skin elasticity, skin hydration, skin radiance, and/or a reduction in fine lines and wrinkles) is improved by at least about 10%, about 20%, about 30%, about 40%, about 50%, about 60%, about 70%, about 80%, about 90%, about 100%, 110%, about 120%, about 130%, about 140%, about 150%, about 160%, about 170%, about 180%, about 190%, about 20%, about 210%, about 220%, about 230%, about 240%, about 250%, about 260%, about 270%, about 280%, about 290%, about 30%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% as compared to prior to the dosing regimen. In some embodiments, the skin property (e.g., skin smoothness, skin elasticity, skin hydration, skin radiance, and/or a reduction in fine lines and wrinkles) is improved by about 10% to about 500% as compared to prior to the dosing regimen. In some embodiments, the skin property (e.g., skin smoothness, skin elasticity, skin hydration, skin radiance, and/or a reduction in fine lines and wrinkles) is improved by about 100% to about 400% as compared to prior to the dosing regimen.

In some embodiments, the disclosure provides a method for reducing wrinkles (e.g., coarse and/or fine lines) at a site on the body of a subject, comprising administering to the subject a dosage form described herein in a dosing regimen, wherein the dosage form comprises an inner capsule and an outer capsule, wherein the inner capsule comprises an HA described herein, the outer capsule comprises a lipid extract described herein, and wherein the inner capsule is contained in the outer capsule. In some embodiments, the site is on the face of the subject. In some embodiments, the site is around the eyes of the subject. In some embodiments, the site is in the crow's feet region of the eyes. In some embodiments, the wrinkles are reduced as compared to prior to the dosing regimen. In some embodiments, the dosing regimen comprises administering the dosage form at least once daily for a duration of at least about 12 weeks, wherein the wrinkles (e.g., wrinkles in the crow's feet region) are reduced by at least about 6 weeks, about 7 weeks, about 8 weeks, about 9 weeks, about 10 weeks, about 11 weeks, or about 12 weeks as compared to prior to the dosing regimen. In some embodiments, the wrinkles (e.g., wrinkles in the crow's feet region) are reduced by at least about 10%, about 20%, about 30%, about 40%, about 50%, about 60%, about 70%, about 80%, about 90%, about 100%, 110%, about 120%, about 130%, about 140%, about 150%, about 160%, about 170%, about 180%, about 190%, about 20%, about 210%, about 220%, about 230%, about 240%, about 250%, about 260%, about 270%, about 280%, about 290%, about 30%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% as compared to prior to the dosing regimen. In some embodiments, the wrinkles (e.g., wrinkles in the crow's feet region) are reduced by about 10% to about 500% as compared to prior to the dosing regimen. In some embodiments, the wrinkles (e.g., wrinkles in the crow's feet region) are reduced by about 300% to about 400% as compared to prior to the dosing regimen.

In some embodiments, the skin property is improved as compared to administering a dosage form comprising the HA or the lipid extract, but not both. In some embodiments, the skin property is improved as compared to administering a dosage form comprising the HA or the lipid extract, but not both, in the same dosing regimen.

### Kits

In some embodiments, the disclosure provides a kit comprising a plurality of dosage forms described herein.

In some embodiments, the plurality of dosage forms is packaged with a scented kit. Said scented insert may be, for example, included in a container with the plurality of solid dosage forms described herein. Including a scented insert may associate a desirable odor with the solid dosage forms or reduce the perception of an undesirable odor, which may increase a subject's compliance with a dosing regimen.

In some embodiments, the insert comprises polymer and an odorant agent. The odorant agent may be derived from natural sources. In some embodiments, the odorant agent comprises a plant oil, one or more compounds derived from plants, or an oil comprising one or more compounds derived from a plant. In some embodiments, the odorant agent is a plant oil, one or more compounds derived from plants, or an oil comprising one or more compounds derived from a plant. In some embodiments, the odorant agent comprises an oil, extract, or scent of berry, cherry, cinnamon, clove, ginger, grapefruit, lemon, lime, nutmeg, vanilla, lavender, or orange, or any combinations thereof. In certain embodiments, the odorant agent comprises a mint oil or mint extract. In one embodiment, the mint is peppermint. In one embodiment, the odorant agent is derived from Mentha piperita L. In some embodiments, the odorant agent comprises vanilla. In some embodiments, the odorant comprises a lemon oil. The scented insert may comprise up to 1%, up to 3%, up to 5%, up to 7%, up to 10%, up to 12%, up to 15%, or up to 20% of the odorant agent by weight. In certain embodiments, the scented insert comprises between 1% to 20%, between 5% to 15%, between 8% to 12%, or between 9% to 11% of the odorant agent by weight. In certain embodiments, the scented insert comprises about 10% of the odorant agent by weight.

In some embodiments, the polymer is a copolymer. The polymer may comprise vinyl acetate. The polymer may be a vinyl acetate copolymer. The polymer may comprise ethylene. In one embodiment, the polymer is ethylene vinyl acetate. The scented insert may comprise up to 99%, up to 97%, up to 95%, up to 92%, up to 90%, up to 88%, up to 85%, or up to 80% polymer by weight. In certain embodiments, the scented insert comprises from 99% to 80%, from 95% to 85%, from 92% to 88%, or from 91% to 89% polymer by weight. In certain embodiments, the scented insert comprises about 90% polymer by weight.

In one embodiment, the scented insert consists essentially of odorant agent and polymer.

The scented insert may be any suitable shape. The insert may generally have the shape of a polygon with three, four, five, six, seven, eight, nine, ten, eleven, twelve, thirteen, fourteen, fifteen, or more sides. The insert may be a triangle; quadrilateral, such as a square, rectangle, rhombus, parallelogram, trapezoid, rhomboid, kite; a pentagon; a hexagon; a heptagon; a octagon; a nonagon; a decagon; a ring; a prism, such as a cube; a sphere; a pyramid, such as a right pyramid, a rectangular pyramid, a rhombic pyramid, or a star pyramid; a cone; a torus; a cylinder; a letter; a symbol; or a word. The insert may be in the shape of a star, for example a three-pointed star, four-pointed star, five-pointed star, six-pointed star, seven-pointed star, eight-pointed star, nine-pointed star, ten-pointed star, eleven-pointed star, twelve-pointed star, thirteen-pointed star, fourteen-pointed star, fifteen-pointed star, or a star with greater than fifteen points. The star may be symmetrical, or may not have a plane of symmetry. The insert may be generally round, for example be circular or ovoid. In some embodiments, the insert is shaped to emulate another object, for example an animal, plant, or inanimate object. In some embodiments, the insert comprises one or more holes or cutouts.

The insert may have a generally consistent thickness, for example having a thickness that deviates less than 10%, less than 5%, less than 2%, less than 1%, or less than 0.5%. In other embodiments, the insert comprises at least one point that deviates greater than 15%, greater than 20%, greater than 25%, greater than 30%, or greater than 35% in thickness from at least one other point. In some variations, the insert is relatively flat. In other variations, the insert is generally not flat. The insert may comprise one or more letters or symbols, for example located on one or more surfaces of the insert. The one or more letters or symbols may be embossed, punched, or etched into the surface.

In some embodiments, the insert is translucent. In some embodiments, the insert is transparent.

In some embodiments, the plurality of dosage forms is packaged in any suitable container. In some embodiments, the container comprises a desiccant. In some embodiments, the container comprises an insert comprising the desiccant. In some embodiments, the container comprises an inner wall, wherein the inner wall comprises the desiccant. In some embodiments, the desiccant is silica. In some embodiments, the container comprises about 1 g, about 2 g, about 3 g, about 4 g, about 5 g, or about 6 g silica.

In some embodiments, the container is, for example, a bottle or a jar. In some embodiments, the container is of any suitable material, such as plastic or glass. In some embodiments, the container comprises a lid, for example a cap, such as a snap-on cap or a screw top cap. In some embodiments, the lid is fully removable, for example, the lid is completely separated from the container. In some embodiments, the lid is connected to the container, for example by a piece of material that is attached to both the cap and the container, such that when the cap is removed from the opening of the container it is still connected to the container.

In some embodiments, the container comprises one or more plastics. In some embodiments, the container is essentially free of one or more bisphenol compounds. In one embodiment, the container is essentially free of bisphenol A. In some embodiments, the container is any suitable color, or may comprise a plurality of colors, or is transparent or translucent. In some embodiments, at least a portion of the bottle is transparent or translucent. In one embodiment, the container is transparent. In some embodiments, the container comprises a bottle and a removable screw-top lid, wherein the bottle and lid comprise plastic and are essentially free of bisphenol A. In some embodiments, the bottle is opaque. In one embodiment, the bottle is blue and the lid is white.

In some embodiments, the container comprises one or more markings, such as a label or design. In some embodiments, the markings comprise one or more words, symbols, instructions, slogans, or combinations thereof. In some embodiments, the one or more markings is any suitable color, or a plurality of colors. In one embodiment, the markings are white.

As described above, in some embodiments the container comprises a scented insert. For example, a scented insert is included inside the bottle, along with the plurality of dosage forms.

In some embodiments, the container is further packaged in one or more additional containers. In some embodiments, the additional container is a box, such as a cardboard box, for example a corrugated cardboard box. For example, in some embodiments the composition is packaged in a container comprising a bottle and a removable screw-top lid, wherein the container is further packaged in an additional container comprising a cardboard box. In some embodiments, the additional container is any suitable color or shape, and may comprise one or more markings. In some embodiments, the additional container is a cardboard box comprising an attached lid that partially tucks into the base, wherein the box comprises a white exterior and a yellow interior, and one or more markings.

In some embodiments, the packaging further comprises instructions, for example one or more pamphlets, sheets, booklets, or other written media. In some embodiments, the instructions are made of any suitable material, such as paper.

Provided herein is a kit, comprising a plurality of dosage forms described herein, and a container. In some embodiments, the kit further comprises instructions. In some embodiments, the plurality of solid dosage forms is packaged in a container. In some embodiments, the container comprises a bottle and a removable screw-top lid, wherein the container comprises plastic. In some embodiments, the container further comprises a scented insert. The scented insert is translucent. In some embodiments, the container is further packaged within an additional container, wherein the additional container comprises a cardboard box. In certain embodiments, the kit further comprises instructions. In some embodiments, the instructions comprise daily administration. In some embodiments, the administration is oral.

### EXAMPLES

### Example 1: Oral Dosage Form for Improving Skin Properties

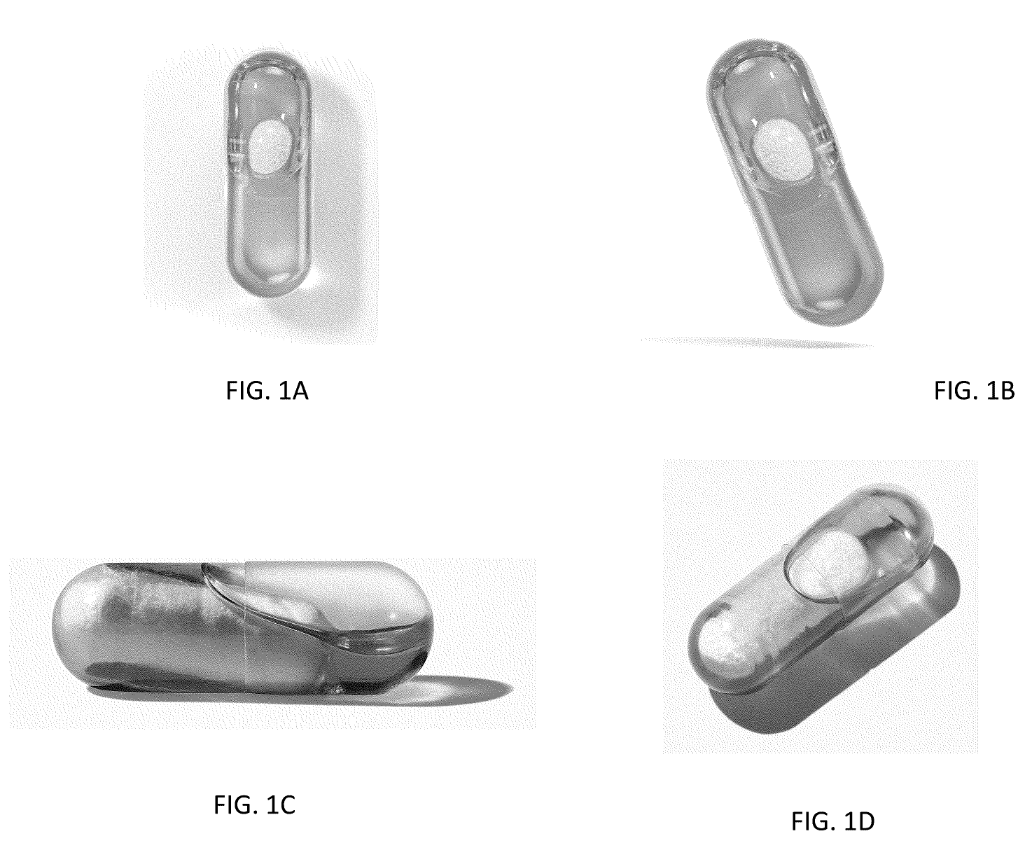

This Example describes a formulation of the HA/WEO supplement for improving skin properties. An image of the exemplary supplement is shown in FIG. 1.

The formulation included two active ingredients: hyaluronic acid and liquid wheat oil extract (Triticum vulgare). The following criteria were considered in designing the dosage form.

The dosage form was selected to contain particular formats of the active ingredients. The hyaluronic acid had an average molecular weight of about 300 kDa. The liquid wheat oil extract had a weight percent of ceramide and DGDG content of ≥2% and ≥15% respectively.

The dosage form was designed to contain a single daily dose of each active ingredient in a single supplement. Initially, a gummy format was designed to formulate the active ingredients. However, it was found that the hyaluronic acid formed gelled pockets when added to a gummy slurry, resulting in homogeneity and manufacturing challenges in forming the gummy dosage form. Additionally, the liquid wheat oil extract was found to mix poorly with the powdered hyaluronic acid. To overcome these challenges, a nested capsule design was developed to keep the active ingredients separate and to avoid exposing the hyaluronic acid with water. The nested capsule was composed of an inner capsule containing the hyaluronic acid in a solid and the outer capsule containing the liquid wheat oil extract in a liquid. This format avoided mixing of the two active ingredients and provided substantially accurate and precise dosing of each.

Further, the inner and outer capsules were formulated as immediate release capsules. It was expected that following oral ingestion and capsule disintegration, hyaluronic acid would contact digestive fluids and form a gel. It was considered that this gelling property would be detrimental to achieving full release if the hyaluronic acid was contained in a delayed release capsule. Without being bound by theory, an immediate release inner and outer capsule is advantageous for ensuring the hyaluronic acid undergoes full release and in regions of the gastrointestinal tract where absorption is optimal.

The dosage form was also formulated to be free of animal-derived components, thus allowing consumption by individuals practicing veganism.

The amounts and formats of ingredients in the supplement are identified in Table 1.

TABLE 1
nested capsule dosage form

| | | | | % of | Input per | |
|---|---|---|---|---|---|---|
| Capsule | Ingredient | mg/serving | Overage % | formula | serving | |
| Outer | wheat extract oil | 350 mg | N/A | 48.86% | 350 | mg |
| | Size 00 | N/A | N/A | 16.75% | 120 | mg |
| | hypromellose | | | | | |
| | capsule | | | | | |
| | Tocomix 70R | N/A | N/A | 0.14% | 1 | mg |
| | (mixed | | | | | |
| | tocopherols) | | | | | |
| Inner | Sodium | 120 mg | 5% | 21.64% | 155.56 | mg |
| | hyaluronate (300 | | | | | |
| | kDa) | | | | | |
| | Size 2 | N/A | N/A | 8.52% | 61 | mg |
| | hypromellose | | | | | |
| | capsule | | | | | |
| | Microcrystalline | N/A | N/A | 3.63% | 26.14 | mg |
| | cellulose | | | | | |
| | Ascorbyl | N/A | N/A | 0.20% | 1.4 | mg |
| | palmitate | | | | | |
| | Silicon dioxide | N/A | N/A | 0.17% | 1.2 | mg |
| | Sunflower oil | N/A | N/A | 0.10% | 0.7 | mg |

The nested capsules were packaged in a bottle containing a scented insert infused with vanilla flavors and medium chain triglycerides. The insert was intended to mask any unpleasant flavors or odors present in the formulation, thereby improving the experience and compliance of the user. As hyaluronic acid absorbs moisture, the bottle was designed to have an inner wall containing about 5.6 grams of silica. This is more silica than can be contained in a standard desiccant packet, and was designed to maintain the interior of the bottle moisture-free.

### Example 2: Oral Dosage Form Provides Improved Skin Properties in Healthy Human Adults

A double-blinded, randomized, placebo-controlled clinical trial was conducted in healthy human subjects with mild to moderate signs of skin aging to evaluate skin condition following daily consumption of the supplement described in Example 1 (“HA/WEO supplement”) for 12 weeks.

### Subject Eligibility

The subjects were male or female with ages spanning 25 to 70 years old. The race, ethnicity and skin type of the subjects varied. The subjects were required to maintain a consistent skincare regimen during the course of the study and to refrain from alternate facial or body treatments. Female volunteers that were pregnant, breastfeeding, or planning to become pregnant were excluded from the study. Further, volunteers with skin conditions on the face, legs, or arms that would interfere with evaluations and/or undergoing treatment for any known health issues or medical conditions were excluded.

### Study Design

Subjects were randomly assigned to receive the HA/WEO supplement or placebo for 12 weeks. 31 subjects were assigned to receive the HA/WEO supplement and 32 subjects to receive placebo. The placebo capsules were visually similar to the HA/WEO supplement and had an outer capsule containing high oleic safflower oil and an inner capsule containing microcrystalline cellulose. Following a baseline assessment by a dermatologist, the subjects were instructed to take one capsule per day for 12 weeks. The participants reported to the testing site for assessment at 4, 8, and 12 weeks.

The outcomes evaluated are identified in Table 2. Assessments used objective measurement devices (Visoscan® VC98 (Courage and Khazaka, Germany) for wrinkles and smoothness, Corneometer® CM 825 (Courage and Khazaka, Germany) for skin hydration, and Cutometer® MPA580 (Courage and Khazaka Electronic, Germany) for skin elasticity), clinical grading by a dermatologist, as well as subjective questionnaires to quantify changes in skin condition and aging parameters. At each time point, the measurements were performed at three sites: face (including cheek and periorbital/crow's feet region), back of left arm, and the left thigh. The measurements were added together across the three sites to provide a global measurement.

TABLE 2
Study Measurements

| | Outcome Measure | Description | Time frame |
|---|---|---|---|
| Primary | Crow's feet | Degree of crow's feet wrinkling | week 0, 4, 8, and 12 |
| | wrinkles | measured via Visioscan ® VC98 | |
| | Skin smoothness | Skin smoothness measured via | week 0, 4, 8, and 12 |
| | | Visioscan ® VC98 | |
| | Skin hydration | Skin surface hydration measured | week 0, 4, 8, and 12 |
| | | via Corneometer ® 825 | |
| Secondary | Skin elasticity | Skin elasticity measured via | week 0, 4, 8, and 12 |
| | | Cutometer ® MPA 580 | |
| Other | Dermatologist | Visual and tactile dermatologist | week 0, 4, 8, and 12 |
| | grading of skin | evaluation of skin condition (fine | |
| | condition | lines in the crow's feet area, | |
| | | wrinkles in the crow's feet area, | |
| | | skin dryness, skin radiance, and | |
| | | skin smoothness) based on 10- | |
| | | point grading scale (with 0 being | |
| | | no signs of aging and 9 being the | |
| | | most severe) | |
| | Self-evaluation of | Self-evaluation of changes in | week 0, 4, 8, and 12 |
| | perceived changes | skin condition and signs of skin | |
| | in skin condition | aging (i.e. fine lines & wrinkles, | |
| | | skin texture, skin smoothness, | |
| | | skin plumpness, skin | |
| | | glow/radiance, skin hydration, | |
| | | skin elasticity, skin redness, skin | |
| | | itchiness) using a 5-point Likert | |
| | | scale (options: much worse, | |
| | | somewhat worse, stayed the | |
| | | same, somewhat better, much | |
| | | better) | |

### Study Outcome

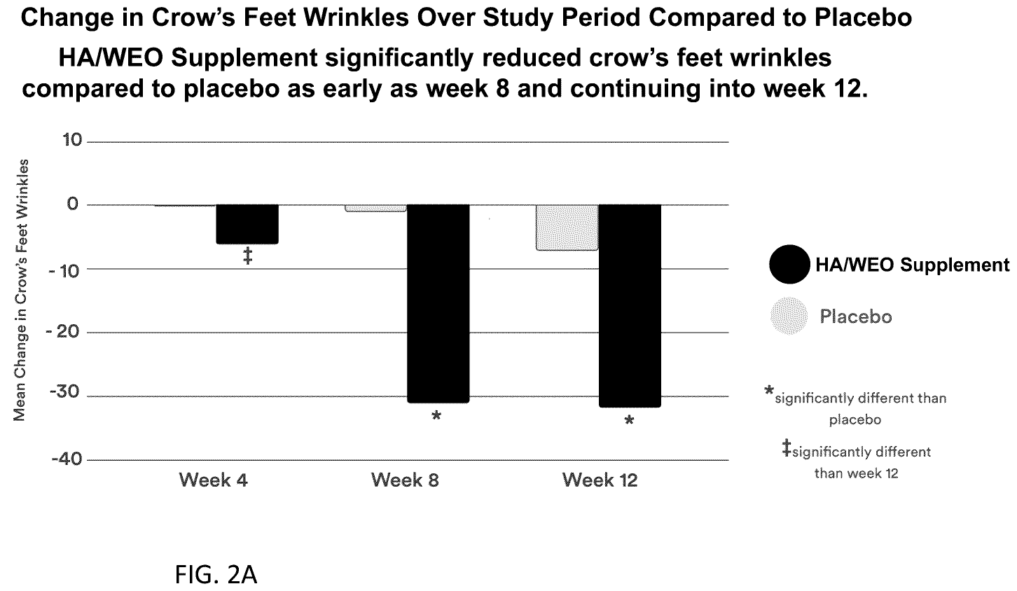

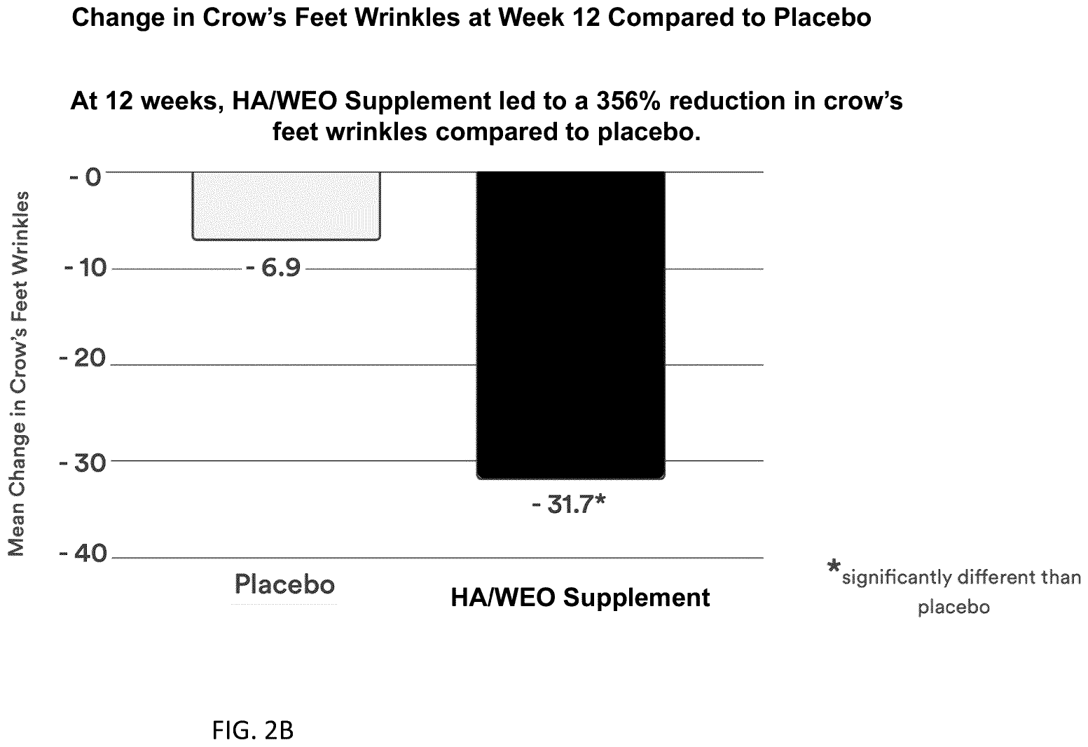

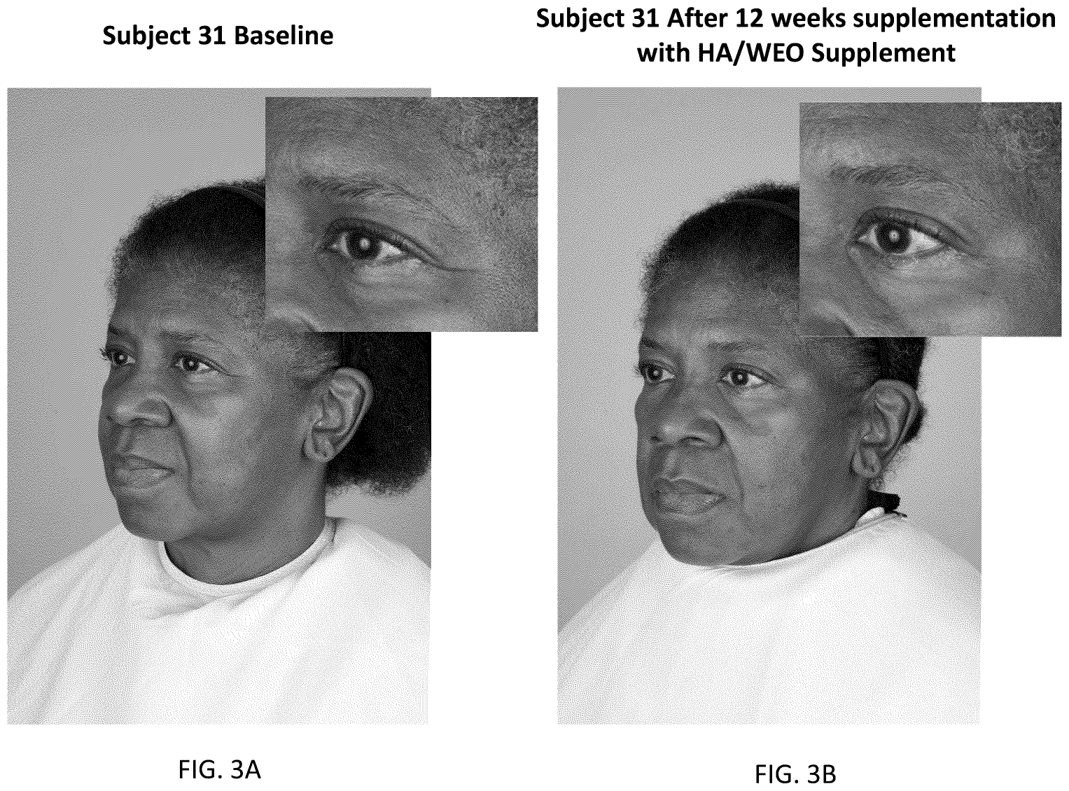

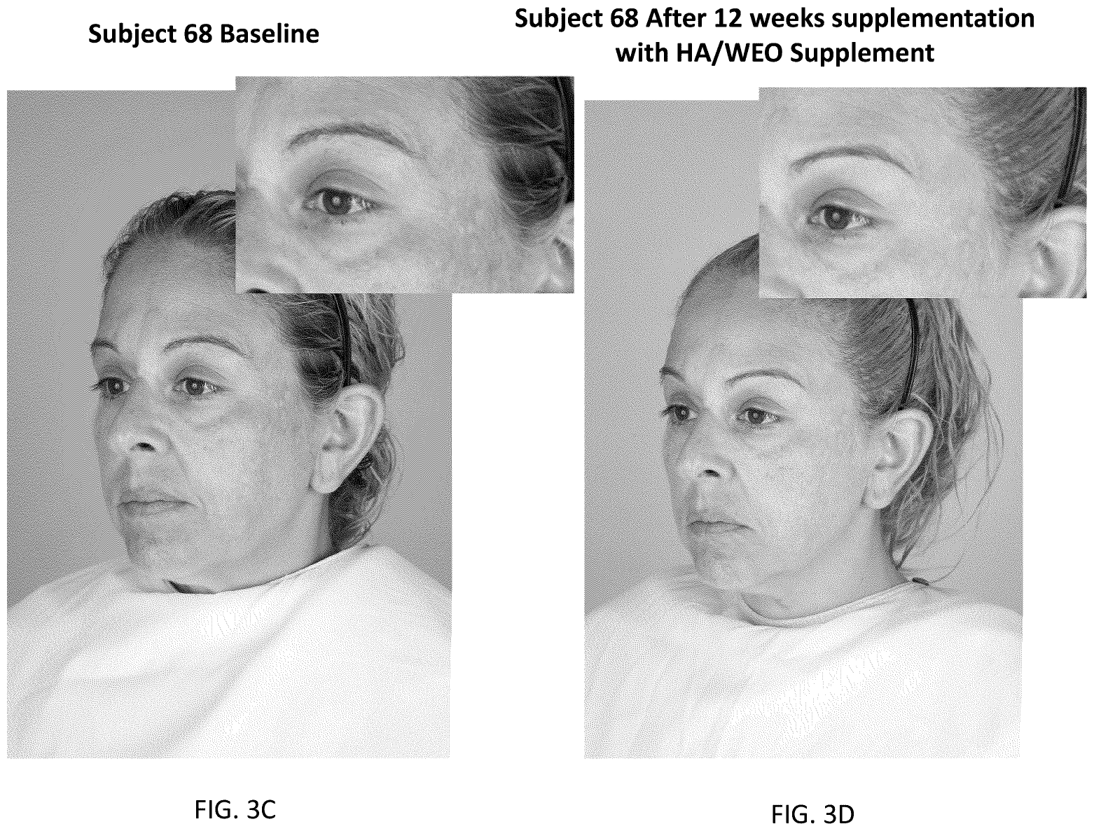

By 8 weeks, subjects that received daily supplementation with the HA/WEO supplement (“test subjects”) experienced significantly reduced crow's feet wrinkles from baseline as compared to placebo (p=0.030) (see FIG. 2A). These improvements remained significant compared to placebo at week 12 (p<0.05). Indeed, as shown in FIG. 2B, supplementation with the HA/WEO supplement led to a 356%, or 3.6×, reduction in crow's feet wrinkles at 12 weeks, compared to placebo. Images of the face of two test subjects at week 12 compared to week 0 are shown in FIGS. 3A-3D. As shown by the inlayed images of the crow's feet regions, visible reduction in wrinkles was observed at week 12 compared to baseline.

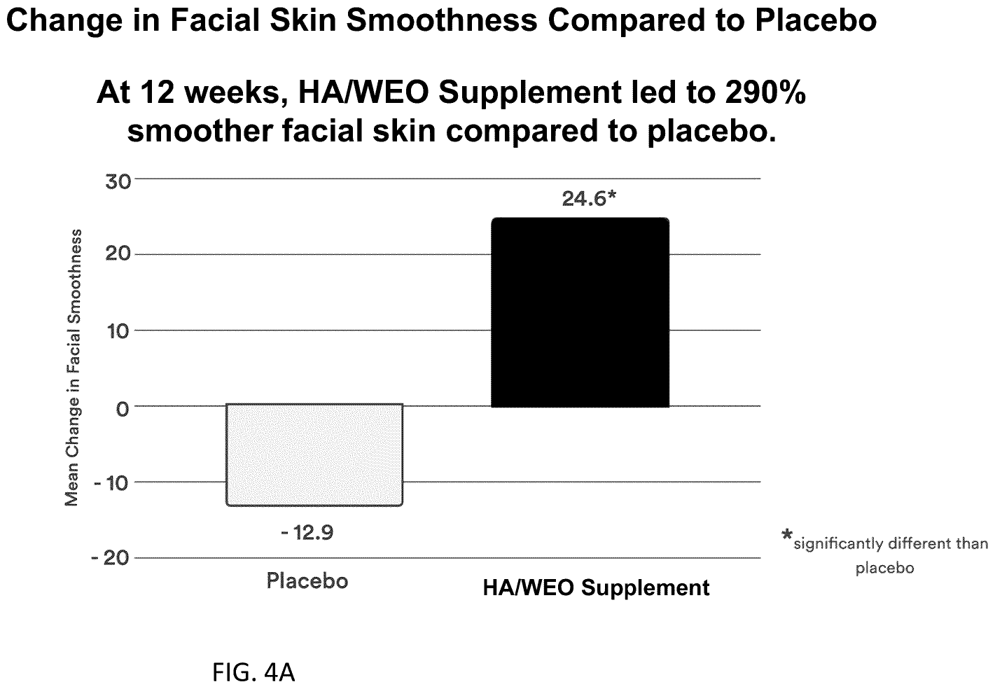

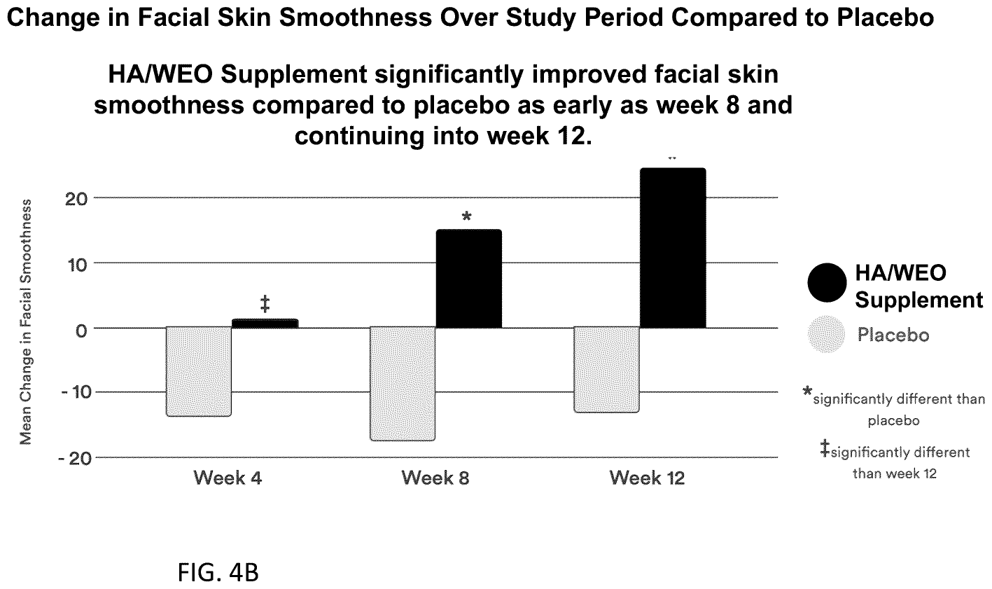

Additionally, by 8 weeks, test subjects experienced significantly improved facial and global skin smoothness from baseline as compared to placebo (p=0.031 and 0.035 respectively). These improvements remained significant compared to placebo at week 12 (see FIG. 4B). As shown in FIG. 4A, supplementation with the HA/WEO supplement led to a 290%, or 2.9×, increase in facial skin smoothness at 12 weeks, compared to placebo.

Additionally, at week 12, the test subjects experienced significantly increased skin hydration and skin elasticity in the thigh region, each from baseline, as compared to placebo (P=0.047 and 0.041, respectively).

Subjective questionnaire data revealed that compared to the placebo group at week 12, significantly more test subjects reported improvement in fine lines and wrinkles, skin radiance/glow, skin resilience/elasticity, and general signs of aging (P=0.017. 0.014, 0.004, and 0.030, respectively).

At weeks 8 and 12, statistically significant improvements in arm skin smoothness, thigh skin smoothness, arm wrinkles, and thigh wrinkles as compared to baseline (p<0.05) were observed. Clinical grading by a dermatologist found statistically significant improvements in arm fine lines, facial skin dryness, facial radiance/luminosity, arm radiance/luminosity, and arm smoothness compared to baseline (p<0.05 for all).

At week 12, statistically significant improvements in face elasticity, global elasticity, arm skin hydration, and global hydration as compared to baseline (p<0.05) were observed.

### Comparison to Individual Active Ingredients

Test subjects in the clinical trial showed improvement in facial skin smoothness as compared to subjects receiving daily supplementation with the individual active ingredients.

In a first clinical study, male and female subjects ages 35-64 years received daily hyaluronic acid supplementation in a placebo-controlled, randomized, double-blind trial (see Hsu, et al (2021) Nutrients 13:2220) (“HA clinical trial”). The supplementation contained 120 mg HA in capsule form, which was administered orally once per day for 12 consecutive weeks. Parameters were assessed at week 0 (baseline), 4, 8, and 12. Facial skin smoothness was measured using Visioscan VC98 (Courage and Khazaka, Germany).

In a second clinical study, female subject ages 45-60 years received oral supplementation with wheat (Triticum vulgare) extract oil daily for 12 weeks in a double-blind, randomized, placebo-controlled clinical study (see Boisnic, et al (2019) J Cosmet Dermatol 18:2027) (“WEO clinical trial”). The supplementation contained 350 mg WEO in capsule form, which was administered orally once per day for 12 consecutive weeks. Parameters were assessed at week 0 (baseline), 4, 8, and 12. Facial skin smoothness was measured using clinical grading by a dermatologist.

The effect on facial skin smoothness of daily hyaluronic acid and wheat oil extract supplementation on facial skin smoothness as evaluated in the HA clinical trial and WEO clinical trial respectively is indicated in Table 3. Comparison is made to facial skin smoothness for test subjects that received a daily HA/WEO supplement.

TABLE 3
effect of daily supplementation of the indicated test
product on facial skin smoothness at week 8 and 12

| | Week 8 | | | Week 12 | | | |
|---|---|---|---|---|---|---|---|
| | Statistical | | | Statistical | | | |
| | significance | | % change | significance | | % change | |
| Test | compared | Effect | compared | compared | Effect | compared | Measurement |
| Product | to placebo | size | to placebo | to placebo | size | to placebo | method |
| HA/WEO | Yes | 0.57 | 187% | Yes | 0.61 | 290% | Visioscan |
| supplement | | | | | | | |
| HA | No | N.D. | N.D. | Yes, but a | N.D. | N.D. | Visioscan |
| | | | | reduction | | | |
| Wheat oil | No | N.D. | N.D. | No | N.D. | N.D. | Clinical |
| extract | | | | | | | grading by a |
| | | | | | | | dermatologist |

N.D. = insufficient data available for calculation

Daily ingestion of the HA/WEO supplement led to a statistically significant improvement in facial skin smoothness from baseline as compared to placebo. This result was statistically significant at both 8 and 12 weeks. Supplementation with wheat oil extract as evaluated in the WEO clinical trial did not lead to a statistically significant improvement in facial skin smoothness at any time point. Supplementation with HA did not lead to a statistically significant improvement in facial skin smoothness at 8 weeks. Supplementation with HA did lead to a statistically significant difference in facial skin smoothness compared to placebo at 12 weeks, however it was a statistically significant reduction in smoothness rather than an improvement in smoothness. In other words, facial skin smoothness in the HA group was statistically significantly worse than in the placebo group after 12 weeks.

1.-41. (canceled)

42. A method for improving a skin property in a human subject, comprising orally administering to the subject a skincare supplement in a dosing regimen of once per day for at least about 8 weeks, wherein the skincare supplement comprises a dose of hyaluronic acid (HA) and a dose of a lipid extract comprising a ceramide and a glycolipid.

43. The method of claim 42, wherein the skin property is selected from
(i) reduced fine lines and/or wrinkles;
(ii) increased skin smoothness;
(iii) increased skin hydration/moisturization;
(iv) increased skin elasticity;
(v) increased skin radiance; and
(vi) a combination of (i)-(v).

44. The method of claim 42, wherein the skin property is improved by about day 56, about day 63, about day 70, about day 77, or about day 84 of the dosing regimen as compared to a first day of the dosing regimen, and wherein the improved skin property is maintained through a duration of the dosing regimen.

45-46. (canceled)

47. A method of improving a dermatological sign of aging in a human subject, comprising orally administering to the subject a skincare supplement in a dosing regimen of once per day for at least about 8 weeks, wherein the skincare supplement comprises a dose of hyaluronic acid (HA) and a dose of a lipid extract comprising a ceramide and a glycolipid.

48. The method of claim 47, wherein the dermatological sign of aging occurs at a site of a body of the subject, wherein the site is present in a facial region, a leg region, an arm region, a waist region, or a combination thereof.

49. The method of claim 47, wherein the dermatological sign of aging is selected from wrinkles, fine lines, dryness, roughness, reduced elasticity, reduced radiance, and a combination thereof.

50. (canceled)

51. The method of claim 47, wherein the dermatological sign of aging comprises crow's feet wrinkles.

52. The method of claim 47, wherein the dermatological sign of aging is improved or not worsened by at least about day 56, about day 63, about day 70, about day 77, or about day 84 of the dosing regimen as compared to a first day of the dosing regimen.

53. A method for improving skin smoothness of a human subject, comprising orally administering to the subject a skincare supplement in a dosing regimen of once per day for at least about 8 weeks, wherein the skincare supplement comprises a dose of hyaluronic acid (HA) and a dose of a lipid extract comprising a ceramide and a glycolipid, and wherein skin smoothness is improved by at least about day 56 of the dosing regimen.

54. The method of claim 53, wherein the skin smoothness is improved by at least about day 63, about day 70, about day 77, or about day 84 of the dosing regimen as compared to a first day of the dosing regimen.

55. The method of claim 53, wherein an improvement to skin smoothness is maintained through at least about day 63, about day 70, about day 77, or about day 84 of the dosing regimen, or through a duration of the dosing regimen.

56. (canceled)

57. The method of claim 53, wherein the skin property is improved at a site of a body of the subject, wherein the site is present in a facial region, a leg region, an arm region, a waist region, or a combination thereof.

58. The method of claim 42, wherein the site is present in (i) a facial region, wherein the facial region comprises an eye, a forehead, a mouth, a nose, a cheek, an ear, or a combination thereof; (ii) a leg region, wherein the leg region comprises a thigh, a calf, a foot, or a combination thereof; (iii) an arm region; and/or (iv) a waist region.

59-63. (canceled)

64. The method of claim 42, wherein the dosing regimen is (i) once per day for at least about 9 weeks, about 10 weeks, about 11 weeks, about 12 weeks, about 13 weeks, about 14 weeks, or about 15 weeks; (ii) once per day for about 8 weeks to about 24 weeks; or (iii) once per day for about 8 weeks to about 12 weeks.

65-66. (canceled)

67. The method of claim 42, wherein the human subject is an adult of about 18 years old to about 65 years old, and exhibits a dermatological sign of aging.

68-70. (canceled)

71. The method of claim 42, wherein the supplement is essentially free of a component derived from an animal.

72. (canceled)

73. The method of claim 42, wherein the HA has an average molecular weight of about 2 kDa to about 800 kDa, about 200 kDa to about 500 kDa, or about 300 kDa.

74. The method of claim 42, wherein the HA is present in a solid, and wherein the dose of HA is about 25 mg to about 240 mg, or about 120 mg.

75-76. (canceled)

77. The method of claim 42, wherein the dose of lipid extract is about 70 mg to about 400 mg, about 300 mg to about 400 mg, or about 310 mg, about 320 mg, about 330 mg, about 340 mg, about 350 mg, about 360 mg, about 370 mg, about 380 mg, or about 390 mg, and wherein the lipid extract is present in an oil.

78-81. (canceled)

82. The method of claim 42, wherein the lipid extract comprises the ceramide and the glycolipid in a weight percent, wherein the weight percent of the ceramide is at least about 2%, and wherein the weight percent of the glycolipid is about 15%.

83. (canceled)

84. The method of claim 42, wherein the glycolipid comprises digalactosyl diglyceride (DGDG).

85. The method of claim 42, wherein the lipid extract is obtained from wheat, optionally from Triticum vulgare.

86. (canceled)

87. The method of claim 42, wherein the skincare supplement comprises a capsule, optionally an immediate release capsule.

88. (canceled)

89. The method of claim 42, wherein the dose of HA and the dose of lipid extract are formulated together or separately.

90. The method of claim 42, wherein the skincare supplement comprises an inner capsule contained in an outer capsule.

91. The method of claim 90, wherein the inner capsule comprises the dose of HA and the outer capsule comprises the dose of lipid extract.

92. The method of any one of claim 90, wherein the inner capsule further comprises an excipient.

93. The method of claim 92, wherein the excipient is selected from microcrystalline cellulose, ascorbyl palmitate, silicon dioxide, sunflower oil, and a combination thereof.

94. The method of claim 90, wherein the outer capsule further comprises an oil.

95. (canceled)

96. The method of claim 90, wherein the inner capsule is a size 2 capsule, and wherein the outer capsule is a size 00 capsule.

97. (canceled)

98. The method of claim 90, wherein the inner capsule, the outer capsule, or both are an immediate release capsule.

99-100. (canceled)

101. The method of claim 90, wherein the inner capsule, the outer capsule, or both is translucent, semi-opaque or opaque.

102. (canceled)
